(19) United States
(12) Patent Application Publication
(10) Pub. No.: US 2024/0252080 A1
(43) Pub. Date: Aug. 1, 2024

(54) MEDICAL DEVICE TO AID PLACEMENT OF A NEEDLE TIP

(71) Applicant: INDOMED LIMITED, Sandyford, Dublin 18 (IE)

(72) Inventors: Owen Laverty, Dublin (IE); Cormac Breatnach

(21) Appl. No.: 18/565,474

(22) PCT Filed: Jun. 1, 2022
(86) PCT No.: PCT/EP2022/064954

§ 371 Date: Nov. 29, 2023

(30) Foreign Application Priority Data
Jun. 1, 2021 (GB) 2107803.5
Jul. 23, 2021 (GB) 2110614.1
Oct. 16, 2021 (GB) 2114811.9
Mar. 28, 2022 (GB) 2204356.6

(51) Int. Cl. A61B 5/154; A61B 5/15

(52) U.S. Cl. CPC A61B 5/1545; A61B 5/150099; A61B 5/150389; A61B 5/150809; A61B 5/150816

(57) ABSTRACT

This invention relates to a medical device for use in aiding the successful placement of a needle tip in a blood vessel. The medical device comprises a body defining a vacuum chamber therein, a port for engagement of a hub of a needle, a fluid passageway through the port from the exterior of the body to the vacuum chamber in the interior of the body, and means to generate an audio/haptic feedback to a user acted upon by a vacuum in the vacuum chamber. The means to generate the audio/haptic feedback comprises an abutment arm and a complementary abutment surface engaged by the abutment arm. The abutment surface has ribs along its length, opposing the abutment arm. The vacuum in the vacuum chamber moves one of the abutment arm and the abutment surface relative to the other, thereby causing the abutment arm to releasably engage a rib to produce the audible/haptic feedback.

### TECHNICAL FIELD

This invention relates to a medical device. More specifically, the invention relates to a medical device for use in aiding the successful placement of a needle tip in a blood vessel.

### BACKGROUND ART

One such medical device is that disclosed in applicants own Patent Co-operation Treaty (PCT) patent application publication No. WO2020/079251. The entire contents of WO2020/079251, including in particular the discussion regarding the problems addressed by these medical devices, the function and the benefits thereof, are incorporated herein by way of reference.

WO2020/079251 discloses a medical device that provides quick and useful feedback to a medical practitioner when they were accessing a central vein during central line insertion. Furthermore, the medical device is less unwieldy than the known devices, simplifies the known techniques for needle placement and is less expensive to manufacture than many of the alternative offerings.

However, it is envisaged that there are a number of aspects of these medical devices that could still be improved upon. For example, it would be advantageous to further improve the speed at which the medical practitioner is alerted to the successful placement of the needle tip inside a vein. This will prevent the medical practitioner from exiting the vein, by passing the needle tip through the rear wall of the vein. Secondly, it would be advantageous to provide feedback that is more frequent and even more noticeable than was heretofore the case. Thirdly, it would be advantageous to provide a medical device that is even more sensitive to detecting when the needle tip has penetrated through the vein wall. Fourthly, it would be advantageous to provide a device that is easier to activate, so as not to move the needle when it is thought to be in situ in a vein or close to same. Fifthly, it is possible to manufacture the device using standard techniques.

It is an object of the present invention to provide a medical device that overcomes at least one of the shortcomings of the known devices, and that provides a useful alternative choice to the consumer.

### SUMMARY OF INVENTION

According to the invention there is provided a medical device for use in aiding the successful placement of a needle tip in a blood vessel, the medical device comprising a body defining a vacuum chamber therein, the body having a port for engagement of a hub of a needle, the port defining a fluid passageway from the exterior of the body to the vacuum chamber in the interior of the body, and means to generate at least one of an audio feedback and a haptic feedback to a user acted upon by a vacuum in the vacuum chamber; and in which the means to generate the feedback comprises an abutment arm and a complementary dedicated abutment surface operably engaged by the abutment arm; the abutment surface having a plurality of closely-arranged spaced abutment members disposed along its length and opposing the abutment arm; the vacuum in the vacuum chamber being operable to move one of the abutment arm and the abutment surface relative to the other of the abutment arm and the abutment surface, thereby causing the abutment arm to releasably engage at least one of the abutment members of the abutment surface to produce the feedback.

By having such a medical device, the means to generate audio/haptic feedback will provide feedback that is more rapid, frequent and noticeable, and therefore easier to detect. This will increase the likelihood that the medical practitioner will recognize the indicator that the tip of the needle is in the blood vessel, and will obviate the likelihood of the medical practitioner passing the tip of the needle through the vein and out the other side of the vein. The abutment members are preferably provided by way of ribs. Alternatively, the abutment members could be provided by way of other protrusions, hollows, indents or an uneven surface for contact by the abutment arm. The means to generate audio/haptic feedback is no longer reliant on a scraper arm passing against the folds of a bladder, which are relatively far apart and require a greater amount of blood to enter into the device to provide feedback to the medical practitioner. This will provide a device that is more sensitive and quicker to clearly indicate that the needle tip is correctly located.

In one embodiment of the invention there is provided a medical device in which the abutment arm and the abutment surface are located internal the body. This is seen as a useful embodiment of the invention. By having the abutment arm and the abutment surface located internal the body, a more compact device is provided.

In one embodiment of the invention there is provided a medical device in which there is provided a valve in the fluid passageway intermediate the exterior of the body and the vacuum chamber operable to selectively apply the vacuum to the port. This is seen as a particularly preferred embodiment of the present invention. By having such a valve, the device may be primed after entry of the needle tip into the body and before entry into the blood vessel. This will ensure that there is no accidental ingress of air into the device with a corresponding loss in vacuum prior to insertion of the needle tip into the patient's body.

In one embodiment of the invention there is provided a medical device in which there is provided a releasable latch operable to control the application of the vacuum to the port. This is also seen as a useful way of priming the device after entry of the needle tip into the patient's body and before entry of the needle tip into the blood vessel.

In one embodiment of the invention there is provided a medical device in which the vacuum chamber comprises an elongate collapsible bellows. Preferably, the bellows is cylindrical, however other shapes such as elliptical, square, rectangular, triangular, pentagonal, hexagonal or other multi-sided in cross-section are readily envisaged. By having an elongate bellows, the bellows may be collapsed in a compact and predictable manner.

In one embodiment of the invention there is provided a medical device in which the collapsible bellows is constructed from resiliently deformable material. This is also seen as a useful aspect of the invention as the bellows will urge to resume its normal shape and this will provide a vacuum force.

In one embodiment of the invention there is provided a medical device in which there is provided a spring operable to extend the collapsible bellows. The spring will further assist in the creation of the vacuum suction force through the port, leading to a more effective device. In addition, the spring will be operable to overcome friction between the abutment arm and abutment surfaces, ensure an even expansion in the bellows, and encourage the bellows to expand in a desired direction.

In one embodiment of the invention there is provided a medical device in which the bellows has a pair of substantially planar opposing end plates. By having planar opposing end plates, the end plates can mate together thereby reducing the amount of air in the collapsed vacuum chamber, leading to a more sensitive device that will react quicker to the ingress of blood into the needle and the resultant change of pressure.

In one embodiment of the invention there is provided a medical device in which the bellows is provided with an internal plug operable to substantially fill the void internal the bellows and minimize the amount of air inside the bellows when the bellows is in a collapsed configuration. Again, by having a plug to fill the void internal the collapsed bellows, the amount of air in the collapsed vacuum chamber will be reduced, leading to a more sensitive device that will react quicker to the ingress of blood into the needle and the resultant change of pressure.

In one embodiment of the invention there is provided a medical device in which there is provided a guide means for the bellows to control the direction of travel of the outermost end of the bellows and by extension the shape of the bellows as it transitions to or from an expanded configuration to or from a contracted configuration. By controlling the manner in which the bellows deforms and re-assumes its at-rest configuration, the device can provide a greater variety, and more controlled, warning notifications than would otherwise be the case. Furthermore, this will obviate the possibility of the bellows becoming “jammed” as it expands back to its at-rest configuration.

In one embodiment of the invention there is provided a medical device in which the bellows is dual skinned with an inner bellows and an outer bellows. This is seen as a particularly beneficial embodiment of the present invention. By having a dual skinned bellows, the vacuum force achieved is greater, the bellows has a much-reduced air pocket when compressed, and the bellows has a reduced internal volume compared to a single skinned bellows having the same external dimensions. The internal bellows effectively forms a plug to reduce the air void. This leads to a more accurate, sensitive device and furthermore will result in a more compact device that is easier to handle by a medical practitioner.

In one embodiment of the invention there is provided a medical device in which the vacuum chamber comprises a releasably detachable vacuum chamber. This is seen as a useful alternative way to provide a vacuum. A container that has been evacuated could be connected to the body of the medical device to provide the vacuum instead of using a bellows. This would enable the magnitude of the vacuum to be accurately selected if desired.

In one embodiment of the invention there is provided a medical device in which there are provided a plurality of abutment arms. By having a plurality of abutment arms, the amount of feedback to the user can be increased, leading to more noticeable warning signs that the needle tip is in the blood vessel.

In one embodiment of the invention there is provided a medical device in which there are provided a plurality of complementary abutment surfaces. In one embodiment of the invention there is provided a medical device in which there is provided a dedicated abutment surface for each abutment arm.

In one embodiment of the invention there is provided a medical device in which the valve is actuated by applying a force perpendicular to the longitudinal axis of a needle connected to the body. In one embodiment of the invention there is provided a medical device in which the latch is actuated by applying a force perpendicular to the longitudinal axis of a needle connected to the body. These are seen as useful as by having the force perpendicular to the longitudinal axis of the needle connected to the body, the needle will not be advanced or retracted by the toggling of the valve or the latch. This will prevent inadvertent over insertion and retraction of the needle, which can greatly improve patient safety.

In one embodiment of the invention there is provided a medical device in which the fluid passageway is defined by a section of the body constructed of silicon rubber for engagement by the valve. The silicon rubber has a relative soft shore A hardness and therefore can be deformed to block off the fluid passageway in the body with relative ease and maintain a vacuum in the bellows. This is seen as useful as by opening the valve with ease the needle will not be advanced or retracted by the toggling of the valve. This will prevent inadvertent over insertion and retraction of the needle.

In one embodiment of the invention the volume of the fluid passageway between the opening of the needle hub and the point of closure of the valve is 0.05 ml. Preferably, the volume of the fluid passageway between the opening of the needle hub and the point of closure of the valve is less than 0.05 ml. The fluid passageway is generally limited in length and diameter to further limit any air voids.

In one embodiment of the invention there is an insert in the fluid passageway either side of the valve to further reduce the effective open cross-sectional area of the fluid passageway to a similar cross sectional area of a needle attached to the device.

In one embodiment of the invention there is provided a medical device in which the vacuum is between 150 mmHg (20 kPa) and 250 mmHg (33.3 kPa). This is seen as particularly suitable for obtaining a quick response from the device and early identification of needle tip placement in a vein.

In one embodiment of the invention there is provided a medical device in which the valve has a shore hardness of between 10 and 55 Shore A.

In one embodiment of the invention there is provided a medical device in which the port is provided with a side port branching off therefrom, the side port having a closure thereon. Again, this is seen as a particularly useful aspect of the invention. The side port may be used for the introduction of wire through the side port, through the port and along the needle allowing the fast execution of the Seldinger or modified Seldinger technique.

### BRIEF DESCRIPTION OF THE DRAWINGS

The invention will now be more clearly understood from the following description of some embodiments thereof given by way of example only with reference to the accompanying drawings, in which:—

FIG. 1 is a perspective view of a medical device according to the invention;

FIG. 2 is an alternative perspective view of the device of FIG. 1 with the bellows and compression spring removed;

FIG. 3 is a cross-sectional side view of the device of FIG. 1;

FIG. 4 is an enlarged view of the circled portion B of FIG. 3; and

FIG. 5 is an enlarged view of the circled portion C of FIG. 3.

FIG. 6 is a perspective view of the medical device similar to FIG. 1 with the bellows in an expanded configuration;

FIG. 7 is a side cross-sectional view of the medical device similar to FIG. 3 with the bellows in an expanded configuration;

FIG. 8 is a view similar to FIG. 2 with the plunger in a retracted configuration;

FIG. 9 is a rear perspective view of a second embodiment of medical device according to the invention;

FIG. 10 is a side cross-sectional view of the medical device of FIG. 9;

FIG. 11 is a side cross sectional view of the medical device of FIG. 9 with the bellows removed for clarity;

FIG. 12 is a rear perspective view of the medical device of FIG. 9 with the bellows removed for clarity;

FIG. 13 is a front perspective view of the medical device of FIG. 9 with the bellows and compression spring removed;

FIG. 14 is a rear perspective view similar to FIG. 13 with the bellows and compression spring removed;

FIG. 15 is a rear perspective view of a third embodiment of medical device according to the invention;

FIG. 16 is a cross-sectional view of the medical device of FIG. 15;

FIG. 17 is an end view of the medical device of FIG. 15;

FIG. 18 is a cross-sectional view along the lines B-B of FIG. 17;

FIG. 19 is a cross-sectional view along the lines A-A of FIG. 17;

FIG. 20 is a rear perspective view of a fourth embodiment of medical device according to the invention;

FIG. 21 is a cross-sectional view of the medical device of FIG. 20 without the valve;

FIG. 22 is another cross-sectional view of the medical device of FIG. 20 without the valve;

FIG. 23 is an enlarged view of the circled portion C of FIG. 22;

FIG. 24 is a cross-sectional view of the medical device of FIG. 20;

FIG. 25 is another cross-sectional view of the medical device of FIG. 20;

FIG. 26 is an enlarged view of the circled portion C of FIG. 25;

FIG. 27 is a front view of a fifth embodiment of medical device according to the invention;

FIG. 28 is a perspective view of the medical device of FIG. 27;

FIG. 29 is a perspective view of the medical device of FIG. 27;

FIG. 30 is a perspective view of the medical device of FIG. 27;

FIG. 31 is an end view of the medical device of FIG. 27;

FIG. 32 is a rear perspective view of a sixth embodiment of medical device according to the invention;

FIG. 33 is a side cross-sectional view of the medical device of FIG. 32;

FIG. 34 is a rear perspective view of a seventh embodiment of medical device according to the invention;

FIG. 35 is a rear perspective view of the medical device of FIG. 34 with plunger depressed;

FIG. 36 is an end view of the medical device of FIG. 34;

FIG. 37 is a cross-sectional view along the lines B-B of FIG. 36;

FIG. 38 is a cross-sectional view along the lines A-A of FIG. 36;

FIG. 39 is a rear perspective view of a eighth embodiment of medical device according to the invention;

FIG. 40 is a perspective cross sectional view of the medical device of FIG. 39;

FIG. 41 is a rear view of the medical device of FIG. 39;

FIG. 42 is a side cross-sectional view along the lines A-A of FIG. 41;

FIG. 43 is a side view of a ninth embodiment of medical device according to the invention;

FIG. 44 is a cross-sectional view along the lines A-A of FIG. 43;

FIG. 45 is a cross-sectional view along the lines A-A of FIG. 43;

FIG. 46 is an enlarged view of the circled portion B of FIG. 45;

FIG. 47 is a rear perspective view of the medical device of FIG. 43;

FIG. 48 is a rear view of the medical device of FIG. 43;

FIG. 49 is a cross-sectional view along the lines A-A of FIG. 48;

FIG. 50 is a cross-sectional view along the lines A-A of FIG. 48;

FIG. 51 is a cross-sectional view along the lines A-A of FIG. 48;

FIG. 52 is a perspective view of a tenth embodiment of medical device according to the invention;

FIG. 53 is a cross-sectional view of the medical device shown in FIG. 52; and

FIG. 54 is a side cross-sectional view of the medical device of FIG. 52.

### DETAILED DESCRIPTION OF THE DRAWINGS

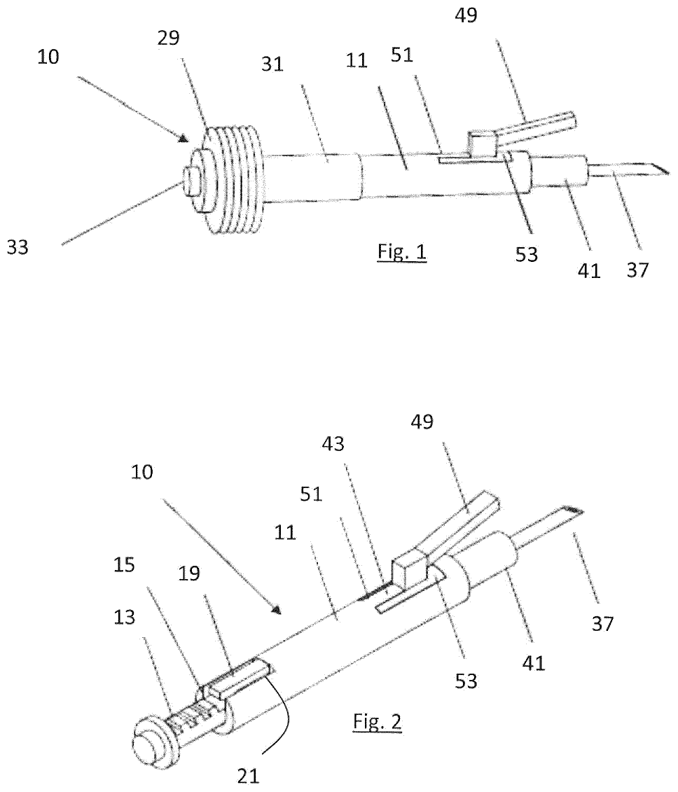

Referring to FIG. 1, there is shown a medical device according to the invention for use in aiding the successful placement of a needle tip in a blood vessel, indicated generally by the reference numeral 10. The medical device 10 comprises a body 11 housing a plunger 13 of circular cross-section, the plunger 13 having raised abutment members, in this instance ribs 15, spaced apart from each other along its length thereby forming an abutment surface. The plunger 13 is located in a channel 17, which is also of circular cross-section, in the main body 11, and the plunger 13 is configured to move in the main body 11, back and forth in a reciprocal fashion in a direction substantially parallel to the main body's longitudinal axis.

The main body 11 has an abutment arm lever 19 adjacent an opening 21. The lever 19 runs parallel to the longitudinal axis of the device 10 with the lever 19 having a protrusion 23 at a free end thereof, the protrusion 23 has a tip 25 which is narrower than a gap 27 between adjacent ribs 15 on the plunger 13. A bellows 29 is provided which in turn has a sleeve 31 which fits over the main body 11, creating an airtight seal due to the interference fit and affixes the bellows 29 to the main body 11.

The plunger 13 is mechanically attached to the bellows 29 at the end 33 of the plunger, and the plunger 13 and bellows 29 are biased away from the main body 11 by way of a compression spring 35. A needle 37 having an inner bore 39 is moulded into the opposite end 41 of the body 11, with the needle's inner bore 39 opening into the inner channel 17 of the main body 11 which houses the plunger 13.

A second lever 43 running substantially parallel to the longitudinal axis of the device 10 is located adjacent the end 41 of the device 10. The lever 43 has a latching surface 45 which engages with a complementary latching ridge 47 on the plunger 13. The second lever 43 extends beyond the outer radius of the main body 11 to form a finger tab 49 which is proximal to the needle 37. The lever 43 is connected to the main body 11 at its base 51 with the opening 53 around the lever 43 filled by over moulding an elastomer into the opening 53. The elastomer adheres to the main body 11 and the lever 43, thereby forming an air tight seal in the opening 53. At the same time, the elastomer allows the lever 43 to flex away from the longitudinal axis of the main body 11 when the finger tab 49 is pulled away from the main body in a substantially radial direction, such that the latching surface 45 no longer engages with the latching ridge 47.

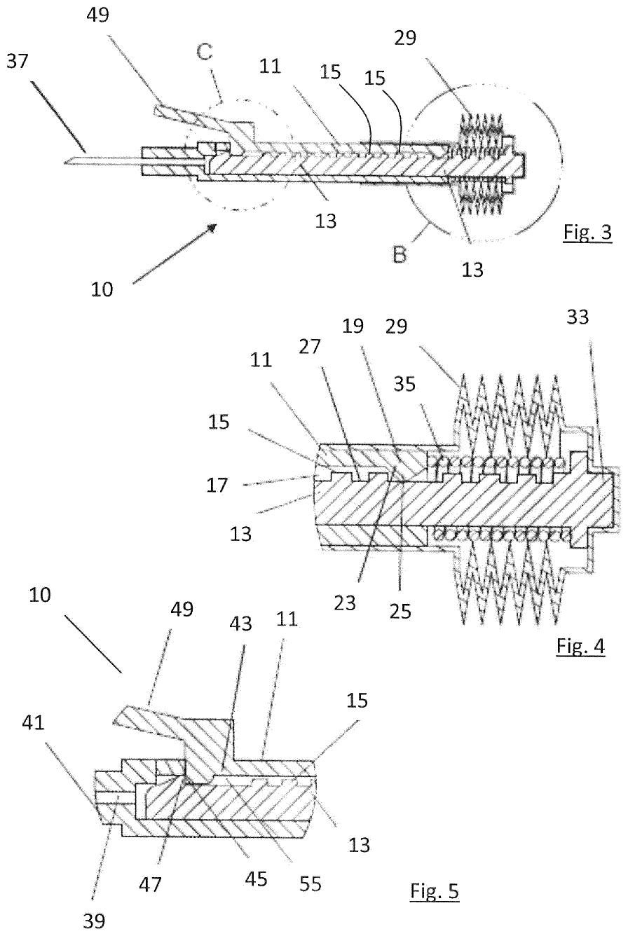

In use, the bellows 29 is compressed (as shown in FIGS. 1, 3 and 4) which in turn compresses the spring 35 and positions the plunger 13 relative to the main body 11 so that the latching surface 45 overlaps the latching ridge 47 of the plunger 13. The latching surface 45 drops into a gap 55 between the latching ridge 47 and one of the ribs 15 on the plunger 13, thereby thus holding the plunger 13 in place and the bellows 29 and spring 35 in compression. The occurrence of the mating of the latching ridge 47 and latching surface 45 provides positive haptic feedback that the device 10 is primed and ready to apply a vacuum to the tip of the needle upon release of the plunger 13.

The operator inserts the needle 37 into a patient and lifts up the finger tab 49. By lifting the finger tab 49, this flexes the lever 43 so that the latching surface 45 disengages from the latching ridge 47, the plunger 13 is released and a vacuum is applied to the needle tip. As fluid enters the needle 37 (once the needle tip is inserted in a vein), the bellows 29 and spring 35 both expand, thereby moving the plunger 13 so that the tip 25 of the protrusion 23 of the lever 19 moves over the ribs 15 on the plunger 13, which in turn causes vibrations and haptic feedback which may be heard and felt by the operator.

It will be appreciated that the device 10 may be provided to the operator pre-compressed so that the operator only has to release the lever 43 once the needle has been inserted into the patient, thereby ensuring a very simple series of operational steps.

While a particular means for retaining the bellows in a compressed state is shown by holding the plunger in its retracted position, other means may be employed such as, but not limited to, squeezing the main body 11 to hold the plunger 13 in position prior to inserting the needle. Indeed, the lever 19 could be pressed by the user to maintain the plunger in place. When the user releases the pressure on the lever 19, the plunger is released and the user with their finger still in contact with the lever 19 will feel the plunger ribs 15 move past the protrusion 23. Indeed, it will be appreciated that there may be a separate portion (not shown) in the main body 11 where the wall is thin, or where there is an opening with an airtight membrane that prevents loss of vacuum in the bellows 29, that will allow a user to feel the plunger 13 through the thinned wall or membrane as the plunger moves in the main body 11 such that it provides haptic feedback. This opening, if provided, could be provided in conjunction with or separate to the lever 19.

As previously described in other embodiments, a spring may not be required depending on the strength of vacuum required at the needle tip and the geometry of the bellows. As described in other embodiments below, the plunger may be formed as part of the bellows thereby eliminating one part.

The advantage of having the needle moulded directly into the main body is that it reduces the amount of trapped air in the device which increases the device's overall sensitivity. It also eliminates the requirement for a needle to be manufactured separately with a luer fitting and flashback chamber. However, if preferred, a needle may not be moulded directly into the main body and instead may be mounted using a luer as described in other embodiments below.

The perpendicular movement of the finger tab 49 to the needles longitudinal axis ensures that the needle is not accidentally advanced or retracted during activation. As described in alternative embodiments, multiple arms (i.e. protrusions 23) and complementary abutment members (i.e. ribs 15) may be employed to increase the sensitivity of the device as well as utilizing the previously described methods for generating haptic feedback.

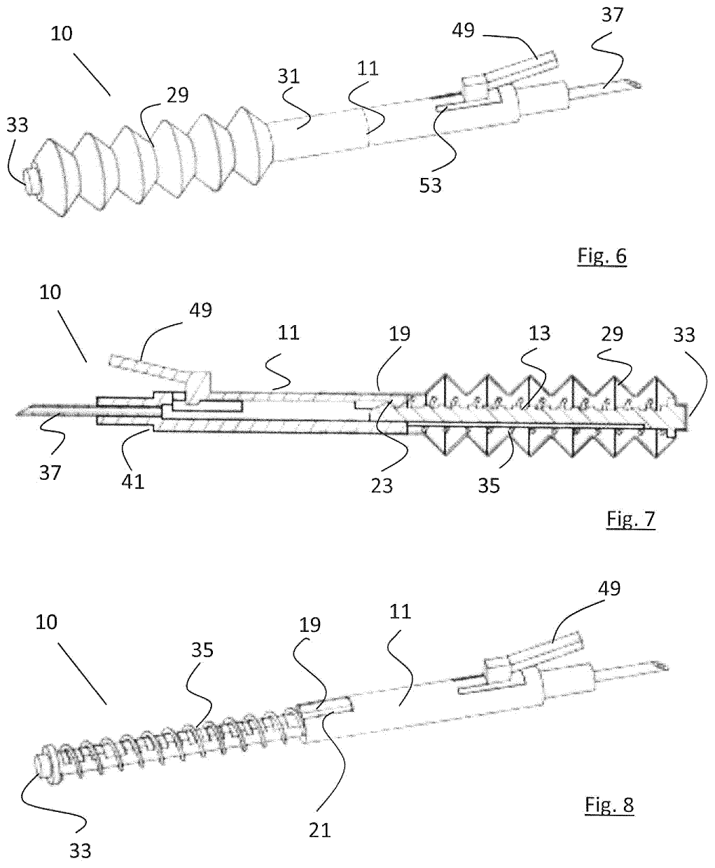

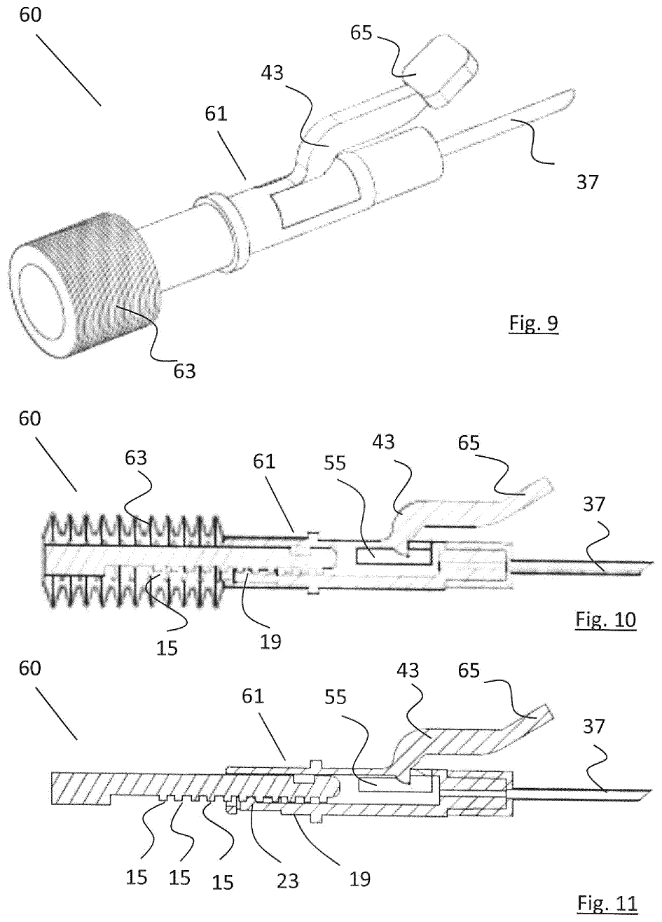

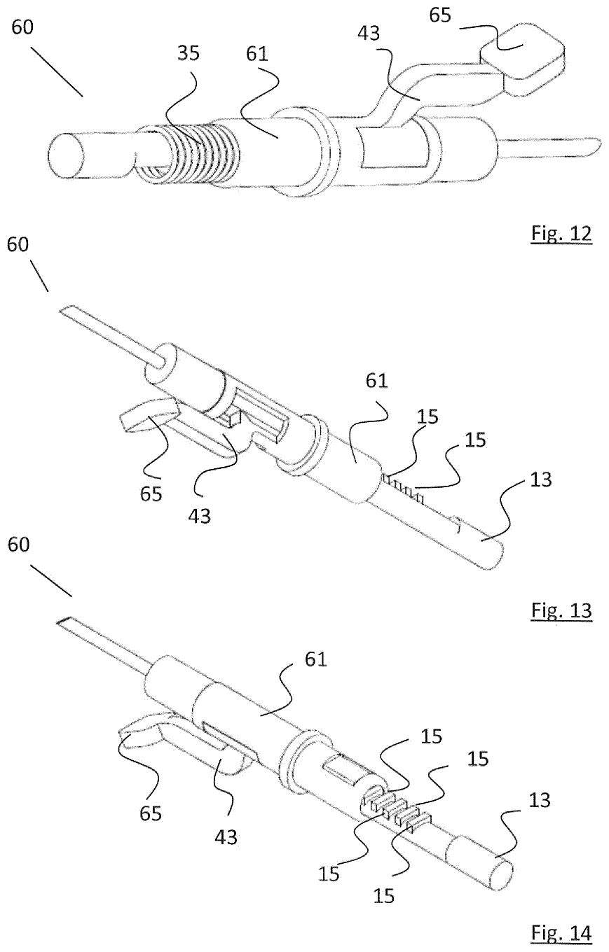

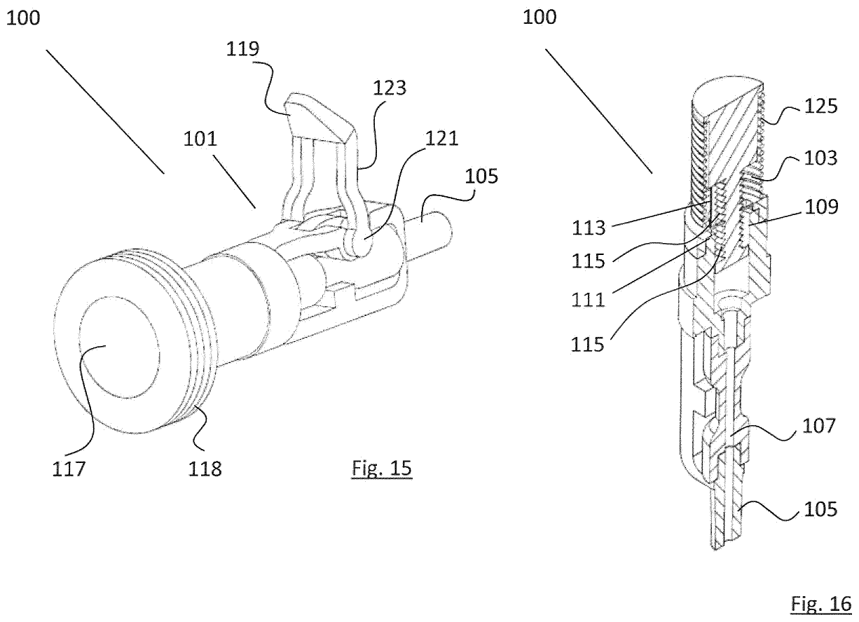

Referring now to FIGS. 9 to 14 inclusive, there is shown an alternative embodiment of medical device, indicated generally by the reference numeral 60. The medical device 60 is similar in construction to the embodiment described with reference to FIGS. 1 to 8 and like parts have been given the same reference numeral as before. The medical device 60 differs from the medical device shown in FIGS. 1 to 8 in that the body 61 of the medical device 60 is shorter than the body 11 of the medical device 10, and the bellows 63 is wider but more compact in a longitudinal direction than the bellows 29 of the previous embodiment. The lever 43 is provided with a finger tab 65 to facilitate release of the lever during operation of the device. In addition, it will be appreciated that previously, the ribs 15 of the medical device 10 were located along an upper surface of the plunger for engagement of an abutment lever arm 19 also positioned along the upper surface of the plunger whereas in the present embodiment, the ribs 15 are located on the underside of the plunger 13 for engagement of an abutment arm lever 19 also located on the underside of the body 61.

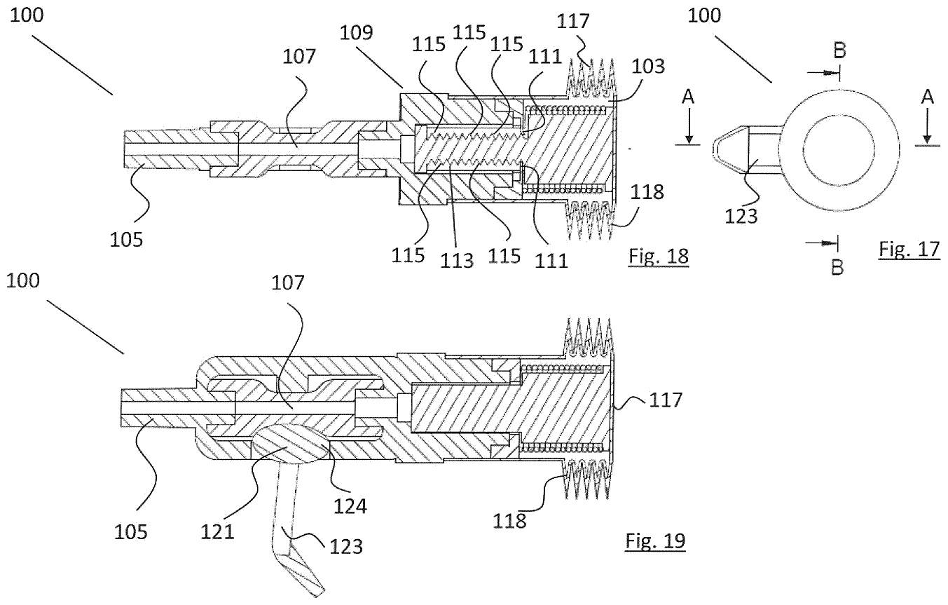

Referring to FIGS. 15 to 19 inclusive, there is shown a third embodiment of a medical device for use in aiding the successful placement of a needle tip in a blood vessel, indicated generally by the reference numeral 100, comprising a body 101 defining a vacuum chamber 103 therein. The body 101 has a port 105 for the engagement of a hub of a needle (not shown), the port 105 defining a fluid passageway 107 from the exterior of the body to the vacuum chamber 103 in the interior of the body.

The medical device is further provided with means 109 to generate an audio/haptic feedback to a user, said means being acted upon by a vacuum in the vacuum chamber. The means 109 to generate audio/haptic feedback comprises an abutment arm 111 and a complementary dedicated abutment surface 113 operably engaged by the abutment arm. The abutment surface 113 has a plurality of closely-arranged spaced ribs 115 disposed along its length and opposing the abutment arm 111.

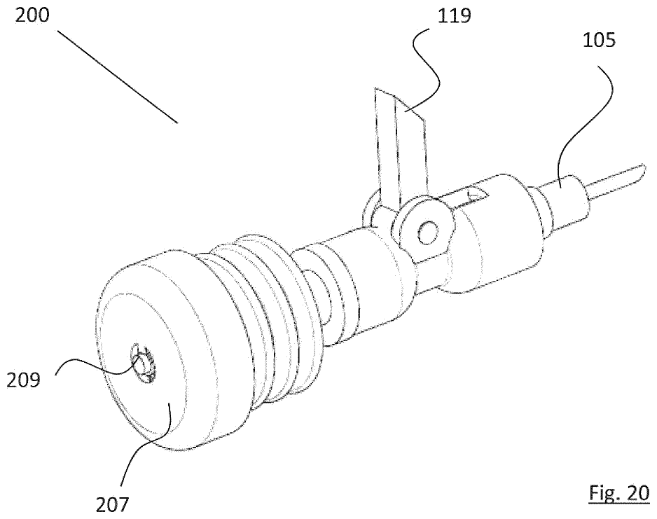

The medical device comprises a plunger 117, incorporating a bellows 118 that may be depressed inwardly, in the direction of the port 105, in order to evacuate the air from the vacuum chamber through the fluid passageway. Once the air has been evacuated from the vacuum chamber, a valve 119 is operated to close the fluid passageway and thereby prevent ingress of air back into the vacuum chamber 103. When the valve is in the position shown in FIGS. 15 and 19, fluid may pass through the fluid passageway however when the valve is rotated approximately 90° about pivot point 121 by manipulating the valve arm 123, an elliptically shaped cam 124 will press inwardly against the fluid passageway 107 thereby sealing the fluid passageway. When the valve has been rotated to a position sealing the fluid passageway, the plunger will remain depressed as fluid cannot get into the vacuum chamber.

In use, air is evacuated from the vacuum chamber by depressing the plunger 117 and then closing the valve 119. A needle (not shown, for a Seldinger technique, or a sheathed needle for a modified Seldinger technique) is connected to the port 105 of the medical device. The tip of the needle is then introduced into the patient, often using ultrasound guidance. When the tip of the needle is inside the patient, the valve 119 is operated to open the fluid passageway 107 once more. As the needle is located in muscle, fluid will not enter into the fluid passageway and the vacuum will remain intact. The medical practitioner may then guide the tip of the needle, using ultrasound guidance if desired, towards the vein. Once the tip of the needle penetrates though the wall of the vein and enters into the blood vessel, blood will be able to travel into the fluid passageway under action of the vacuum in the vacuum chamber 103. As the assembly is closed to the atmosphere, hazardous entrainment of air into the bloodstream is prevented.

As the blood enters into the fluid chamber, the plunger and bellows that were previously held in the contracted configuration by the vacuum in the vacuum chamber will begin to expand outwardly in a direction away from the port 105. This expansion is caused in part by the elastic properties of the resiliently deformable material that the bellows 118 is constructed from, and in part by a helical spring 125 (seen most clearly in FIG. 16 in which the bellows 118 has been removed for clarity). It is envisaged that either the natural resilience, or the spring, would be sufficient to provide this force, however the combination of the two provides for quicker and more sensitive operation of the device. It will be appreciated however that a spring may not necessarily be employed. When a spring is employed it will be appreciated that the bellows can be pushed beyond its fully contracted/compressed shape by the force of the spring on closure of the valve and priming of the device which is not shown in the drawings thereby generating a higher vacuum than would be possible by using a bellows alone. This applies to all of the embodiments using a bellows and spring combination described below to generate a vacuum.

As blood enters into the fluid chamber, drawn inwardly by the vacuum in the vacuum chamber 103, the abutment surface 113 will be moved relative to the abutment arm 111, thereby causing the abutment arm 111 to releasably engage at least one of the ribs 115 of the abutment surface 113. As the abutment arm comes into contact with the rib 115, it produces the audible/haptic feedback. As more blood enters into the fluid passageway and up into the bellows, the abutment arm 111 will come into contact sequentially with the spaced apart ribs along the length of the abutment surface, causing a series of clicks as the abutment surface moves relative to the abutment arm.

It can be seen that in the embodiment shown, there are two abutment arms 111, one on either side of the abutment surface, and there are a set of ribs 115 on both, opposing sides, of the abutment surface. More or less than two abutment arms could be provided and indeed more than one abutment arm could be provided on one or more set of ribs if desired. The two abutment arms and the two sets of ribs are offset with respect to each other in the direction parallel to the longitudinal axis of the medical device. In this way, the abutment arms and associated ribs will alternate when they provide the audible/haptic feedback. Preferably, the two sets of ribs are offset by half the distance between the peaks of a pair of adjacent ribs 115.

Various advantages of the embodiment will become apparent from further inspection. For example, in order to ensure the first click of the abutment arm against a rib happens as quickly as possible, it is advantageous if the abutment arm is inclined to move as quickly and as forcibly as possible in response to a pressure change in the bellows. In the embodiment shown in FIGS. 15 to 19, there is shown a layout whereby a spring 125 biases the plunger 117 against the bellows 118 end. The plunger has ribbed surfaces 115 patterned along the plunger's length which are opposite each other and a half pitch out of position with each other along the plunger's length.

The spring 125 overcomes the resistance of the abutment arm/ribs ensuring that the required vacuum is generated. The spring increases the speed at which the bellows 118 expands, imparting more expanding energy to the bellows resulting in a higher vacuum which draws fluid to flow into the needle and bellows more quickly which provides for an earlier first and subsequent clicks. The force that the spring 125 generates that is (preferably) over and above that which is necessary to overcome the friction of the abutment arm over the ribs increases the vacuum force generated by the bellows. In this way, the spring and bellows combination creates a more responsive device speeding up the commencement of the haptic/acoustic feedback.

The springs expansion and therefore the plunger movement and generation of feedback is restrained by the bellows being held by the vacuum. Therefore, while the abutment arm is no longer on the bellows as with the previous implementations, its movement is still directly controlled by it. Importantly, the spring 125 and the plunger 117 arrangement limits/prevents a concave surface being formed when the bellows flat end is depressed to create a vacuum. This is advantageous as it obviates the need for fluid ingress to first act on the concave surface to reform a flat surface at the outermost end of the bellows 118 before the bellows can begin to expand longitudinally. It also directs the expansion of the bellows 118 along the longitudinal axis of the medical device therefore limiting the bending of the bellows, and this can be further supported by providing a mating feature between the bellows and the plunger 117 to allow the plunger to locate on the bellows.

The plunger 117 advantageously hits a stop when fully pushed into the body 101, indicating that the bellows has been fully displaced and is generating its maximum vacuum, the stop providing feedback to the user that the bellows has been fully compressed and the maximum amount of air has been expelled from it prior to the valve being closed priming the device. This provides comfort to the operator that the device is primed and makes the device more usable and provides repeatable performance from the device. On releasing the force compressing the bellows, the bellows rebounds, preferably bending the abutment arm against the ribs priming it for a very small movement of the bellows on a small volume increase. In relation to the overall dimensions of the medical device, the configuration shown in FIGS. 15 to 19 inclusive enables a bellows 118 with a very small internal volume/diameter to be manufactured to enable a relatively long travel of the plunger for any given pressure/volume change caused by blood entering the needle. Additionally, the plunger reduces the air pocket in the compressed bellows, effectively forming a plug and reducing the effective compressed internal volume of the bellows, thus making it more responsive to pressure changes.

Another advantageous aspect of the present invention is the manner in which the abutment arm and the abutment surface are both located internal the bellows and the body of the device. Having all these components internally advantageously reduces the air pocket and provides for a more ergonomic solution than having a spring externally mounted. Having the spring 125 and the means 109 to generate audio/haptic feedback located internally removes the need for a bellows housing which would add thickness and weight to the device, and allows for additional haptic feedback of the bellows moving in the operators hand and allows the operator to see directly into the bellows. Advantageously, this embodiment allows for the easy removal of the bellows from the valve for blood sampling. It will be understood that this feature of easy removal of the bellows, although useful, is not essential. Instead, the operator could simply compress the bellows once more to express a blood sample from the device.

The loaded spring plungers 117 movement is controlled by the pressure in the bellows 118 with the spring 125 enabling a higher vacuum, stronger rebound, and therefore faster response once a blood vessel has been punctured. The valve can be over moulded into the main body reducing the number of assembly steps. The valve is preferably formed of a thermoplastic elastomer (TPE) that can be over-moulded onto the main body of the device which is preferably made of transparent grade Polypropylene, a high density polyethylene (HDPE), or a medical grade Silicone rubber with a shore hardness of between 10 and 55 Shore A. Thin plastic or metal clicker material could be employed for a clicker (abutment arm) with the plastic preferably having a minimum shore hardness of 60 Shore D. A similar shore hardness is used in the plunger and abutment members (ribs) to provide an audible click which should be audible even when immersed in blood.

The spring loaded plunger can contribute more vacuum in the bellows than the bellows itself, particularly where the bellows is of a softer material. Indeed, the plunger can have walls/a sleeve that prevents the deformation of the bellows walls as it is forced to expand under the force of the spring, ensuring an adequate vacuum is generated. This is particularly relevant in those cases where the bellows walls cannot maintain their intended shape while extending due to a high vacuum level created by a spring.

A bellows having a volume in the range of 0.5 ml to 2.5 ml may have limited ability to force the abutment arm over the abutment surfaces. The addition of a spring ensures that the abutment arm moves over the abutment surface, supplementing the bellows.

While the ribs 115 are shown on the plunger 117 it will be appreciated that the ribs 115 may be formed in the fluid passageway 107 and indeed these may be formed by openings to facilitate moulding (not shown) with the abutment arms 111 located on the plunger 117, Advantageously the neck of the bellows 118 covers these openings to maintain an air sealed device.

Referring now to FIGS. 20 to 26 inclusive, there is shown a fourth embodiment of medical device according to the invention, indicated generally by the reference numeral 200, where like parts have been given the same reference numeral as before. The medical device 200 differs from the previously described medical device in that it contains a twin-skinned bellows 201 comprising an outer bellows skin 203 and an inner bellows skin 205. A plunger end cap 207 is connected to the outermost ends of each of the outer bellows skin 203 and the inner bellows skin 203. The plunger end cap 207 has an aperture 208 for through passage of an elongate shaft 209.

Referring specifically to FIGS. 23 and 26, it can be seen that there is an abutment arm 211 on the plunger end cap 207 and there is an abutment surface 213 on the elongate shaft. The abutment surface is again provided by way of a plurality of closely spaced-apart ribs 215. By closely, what is meant is less than 1.0 millimeter (0.001 m) apart. In some embodiments, the spaced-apart ribs may be more than 1.0 millimeters (0.001 m) apart, but less than 4.0 mm (0.004 m) apart. In fact, once again, there are two abutment arms 211 and a pair of abutment surfaces 213, one for each of the abutment arms and each of the abutment surfaces 213 containing a plurality of ribs along its length for engagement of a complementary abutment arm 211.

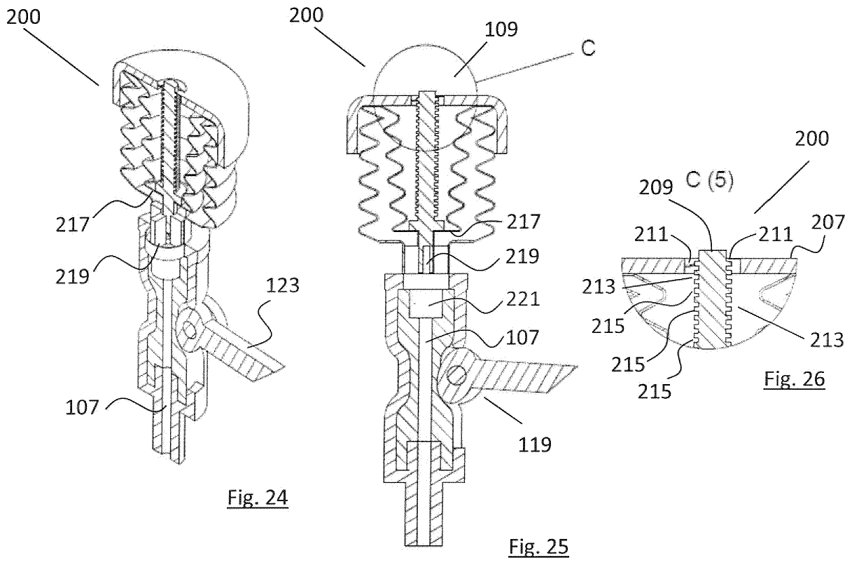

In use, the end cap is depressed in order to evacuate air from the bellows 201, and the elongate shaft 209 passes through the aperture 208 in the plunger end cap 207. Once the air has been evacuated, the valve 119 (as shown in FIGS. 24 and 25) is operated to close off the fluid passageway 107 and maintain the bellows in a collapsed state. A needle or sheathed needle is attached to the port 106 and the needle tip is inserted into the patient before the valve is released once more. Once the needle tip enters the blood vessel, the vacuum force created by the natural resilience of the bellows 201 will cause the bellows to expand and the blood to be drawn into the bellows. As the bellows 201 expands, the plunger end cap 207 will move outwards, causing the abutment arms 211 to move against the ribs 215 of the abutment surface 213, thereby providing the audible/haptic feedback to the user that is indicative that the tip of the needle is correctly positioned in the vein.

There are several additional features and advantages to the construction described in FIGS. 20 to 26 inclusive. For example, the double bellows is shown with an external folded wall 203 and an internal folded wall 205 with a central ribbed shaft 209 attached to the base of the internal bellows. The ribbed shaft 209 runs through an aperture in the plunger end cap 207 which in turn is provided with abutment arms. The end cap sits on the end of the bellows and moves with the bellows as it expands and contracts generating haptic and audio feedback.

A double bellows with external diameter of 13 mm and internal diameter of 6.4 mm can generate twice the expansive force with 75% less volume than the equivalent single bellows with an external diameter of 13 mm for the same material and wall thickness. This reduction in volume correlates to an increase in length along its axis with any pressure increase in the bellows and a corresponding increased length change. Advantageously, with its doubled-up walls it can generate nearly twice the vacuum of a standard single bellows of the same external diameter. This arrangement allows for an in-line abutment arm and rib arrangement, obviating the risk of the bellows being prevented from moving during expansion due to jamming between the ribs and arms.

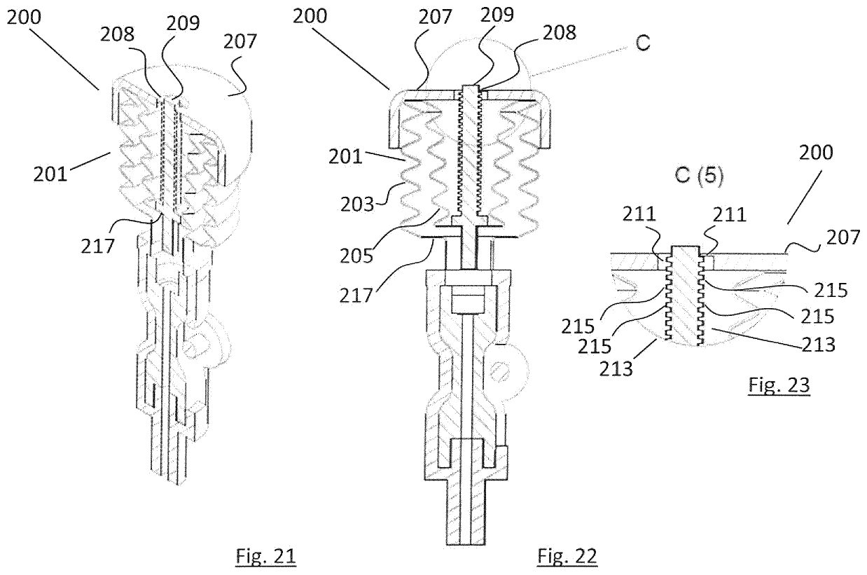

The central ribbed shaft 209 is attached to the base 217 of the inner bellows and extends outwardly beyond the base of the inner bellows. This base 217 of the inner bellows forcibly inserts into a space created by a cruciform 219 (as best illustrated in FIG. 24) of a valve connector 221, with the valve connector 221 creating a seal with the opening of the bellows 201 such that when inserted into the bellows 201 there is only a hole connecting the bellows internal volume with the fluid passageway 107 of the valve housing, the valve connector 221 having a push fit with the main valve body (this valve connector 221 component has been removed from FIGS. 21 and 22 for ease of reference of the remaining components).

Advantageously the central shaft 209 with ribs 215 connecting the base of the inner bellows with the base of the outer bellows ensures that the walls of the inner and outer bellow sections move in unison applying the larger expansive force to the bellows and the plunger end cap 207.

Of particular note is the abutment arm clicker which is located in the plunger end cap with two abutment arms being ½ pitch out of spacing to allow for an improved level of sensitivity. The number of abutment arms could be increased with the pitch changed accordingly. This aspect of the present invention can be applied to most if not all of the embodiments described herein.

Referring to the exploded views the extended portion of the inner bellows extends through the major opening of the bellows and locates in between the ribs of the cruciform on the neck part. The ribbed shaft 209 forcibly locates in the opposite side of this extended portion from the open side, firmly connecting the valve connector 221, bellows 201 and ribbed shaft 209 together. Advantageously the cruciform ribs 219 arrangement on the valve connector 221 allows for air to flow centrally through the central opening of the neck part, between the cruciform ribs 219 and into the bellows 201 internal volume.

It will be understood that this arrangement allows for an increase in the maximum potential vacuum generated in a given outer diameter of cylindrical bellows, reduces any air pockets while compressed, increases the responsiveness of the system and keeps the abutment arms and ribs of the abutment surfaces along the central axis. The design also allows for a hard stop for the operator to provide comfort. The double bellows could be made in more than one part to facilitate manufacturing.

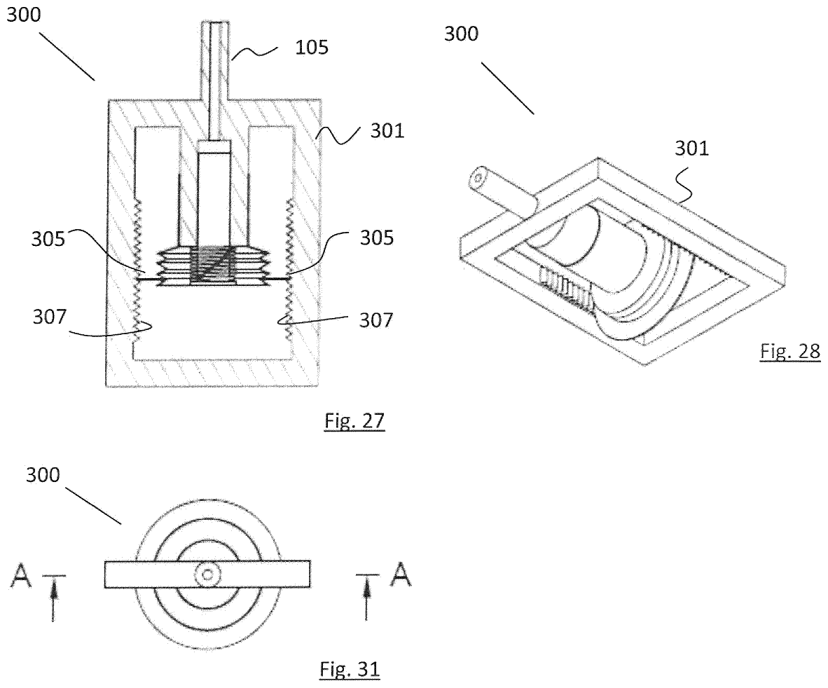

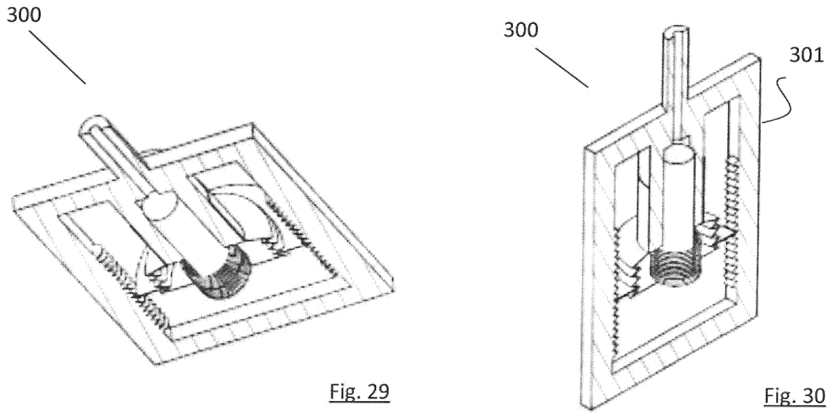

Referring now to FIGS. 27 to 31 inclusive, there is shown a fifth embodiment of medical device according to the present invention. It can be seen that the medical device comprises a substantially external rectangular frame 301 for guiding the plunger 302 and the bellows 303. A pair of abutment arms 305 are mounted on the plunger 302 and a pair of abutment surfaces 307, each having a plurality of ribs, are formed on the inside faces of the sides of the frame, opposing the abutment arms.

In use, the plunger is depressed and the bellows is contracted to evacuate air out of the bellows. A needle is connected to the port 105. A valve (not shown), if provided, is sealed to prevent early expansion of the bellows. Alternatively, if the valve is not provided, the plunger is held in position by the user keeping their thumb on the plunger. Alternatively, a latch (not shown), if provided, could be used to prevent early expansion of the bellows. Once the needle tip has entered the body, the plunger may be released as it will not expand until the needle tip enters a blood vessel.

There are several advantages of this embodiment. This is a very simple embodiment with a two piece part (excluding a valve) where the abutment arm is formed as part of the bellows (although it may be separate). In use, the bellows 303 is compressed and the valve closed before the attached needle (not shown) enters the body at which stage the valve is opened. The integrated abutment arm has openings which align with the arms of the frame, the oppositely facing surfaces presenting ribbed surfaces which slightly overlay and engage with the contacting edges of the abutment arms. This arrangement allows for a bellows with a relatively small diameter and therefore provides a high level of movement for any volume change. A spring could be applied internally or externally as previously described. It is envisaged that the opening of the bellows 303 would need to be larger to facilitate a spring being located internally.

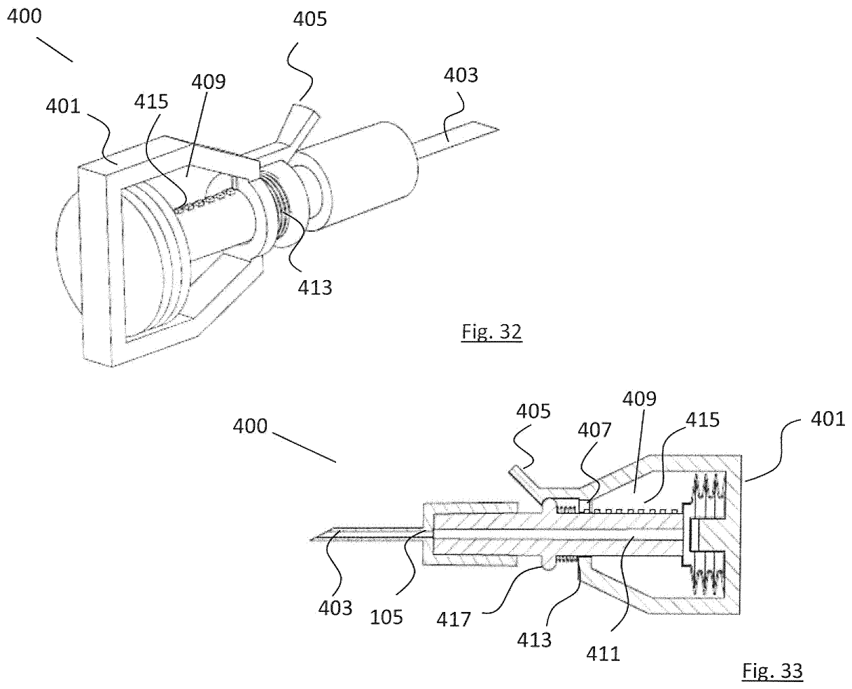

Referring now to FIGS. 32 and 33, there is shown a sixth embodiment of the present invention, indicated generally by the reference numeral 400, and where like parts have been given the same reference numeral as before. The medical device 400 comprises a frame 401 with a port for the attachment of a needle 403. The frame 401 has an opening which the stem of the bellows 407 loosely fits through, with the other end of the bellows being attached to the frame which is tightly mated with an inner protrusion in the bellows. A spring 413 biases the frame from the bellows stem. The frame has a clasp 405 for engaging the bellows 407 and holding the frame in position on the bellows stem with the bellows in a collapsed configuration. When it is desired to use the device, and the needle tip is located in the body of the patient, the clasp 405 may be released. As the bellows 407 expands with additional force provided by the spring, it moves the frame 401 and an abutment arm on the frame will move along and against the abutment surface 409 on the outer surface 411 of part of the bellows clicking on the ribs 415 on the abutment surface 409.

In this embodiment, no valve is required. The user releases the contracting force on the bellows when the needle is inserted into the patient, allowing it to expand when the clasp is released thus creating a vacuum in the bellows. The protrusion in the bellows that fits on the frame reduces the air pocket in the bellows, increasing sensitivity. The clasp 405 or the abutment arm could be designed to break, limiting its use to a single use device. The ribs 415 and the flange 417 on which the clasp locks onto may be made from a separate, third part that fits over the bellows stem.

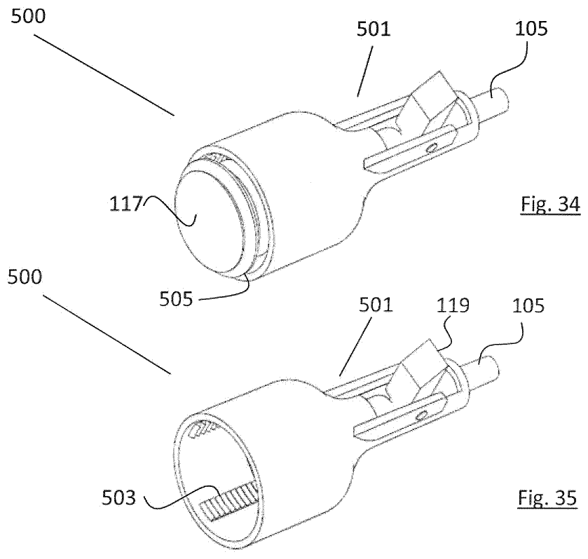

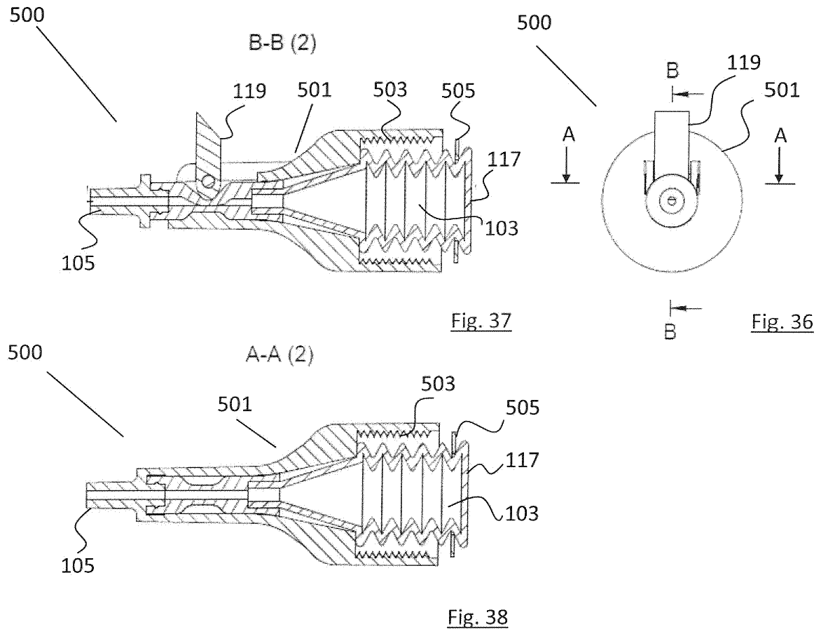

Referring now to FIGS. 34 to 38 inclusive, there is shown a seventh embodiment of medical device according to the present invention, indicated generally by the reference numeral 500, in which like parts have been given the same reference numeral as before. The seventh embodiment is similar in construction in many ways to the third embodiment (described with reference to FIGS. 15 to 19 inclusive above). Referring first of all to FIGS. 34 and 35, there is shown a medical device having a body 501, a plunger end cap 117, a valve 119 and a port 105. In FIG. 34, the device is at rest and the plunger end cap 117 has not been depressed and the bellows (not shown) has not been compressed. In FIG. 35, the user has pressed the plunger end cap 117 inwardly with their thumb, thereby compressing the bellows. Once the bellows (not shown) has been compressed and the air expelled from the bellows, the valve may be operated to seal the fluid passageway 107. Referring specifically to FIG. 35, when the plunger end cap 117 has been depressed, the ribs 503 of an abutment surface are visible. These ribs 503 engage against an abutment disc 505 on the bellows.

Referring specifically to FIG. 37, it can be seen that the valve 119 has an alternative cam surface. When the valve arm 123 is in the upright position as shown in FIG. 37, the fluid passageway 107 is blocked off. If the valve arm is pivoted about pivot point 121, forward or backwards by the user manipulating it with their finger or their thumb, the valve will open up the fluid passageway once more. It will be understood that the valve arm may be positioned closer to a needle luer attached to the device, similar to the third embodiment illustrated in FIGS. 15 to 19 above. In that way, the user may release the valve arm in a direction substantially perpendicular to an attached needles longitudinal axis.

In use, the plunger end cap is depressed inwardly into the body and the valve is put into the closed position, as illustrated in FIG. 37 (it will be understood that in this configuration, the bellows 103 has not been collapsed and evacuated of air, and the plunger end cap 117 has not been pressed inwardly. Only the valve has been manipulated). When the plunger has been pressed inwardly to collapse the bellows, the valve is closed and a needle is placed on the end of the port 105. The needle is then introduced into the patient's body before the valve is released/opened. When the needle tip (not shown) enters the blood vessel, the bellows 103 will be free to expand once more under its own elastic force of the resiliently deformable material from which it is formed. An abutment arm 505 mounted on the outermost end of the bellows contacts a plurality of ribs 503 of an abutment surface on the inside of the body as the bellows 103 expands, producing the audible and/or the haptic feedback. Although shown as a separate part, the abutment arm could be formed as part of the bellows 103.

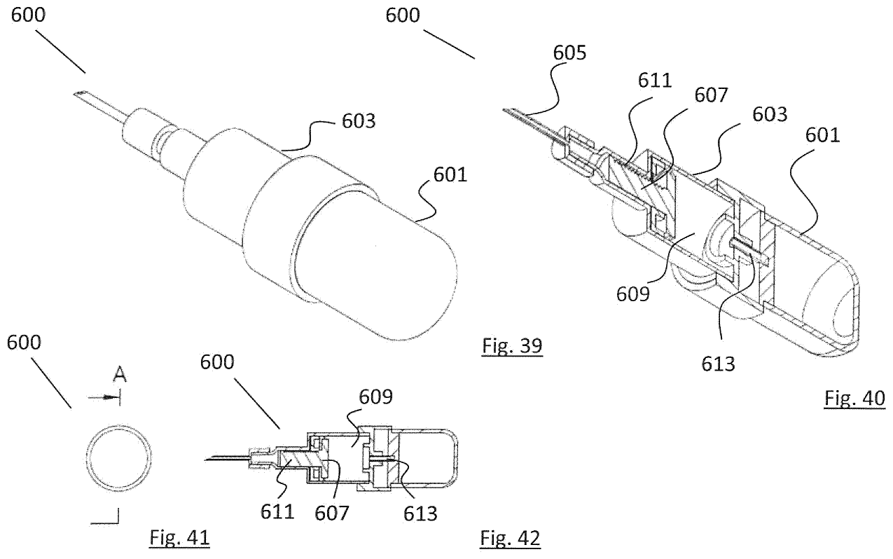

Referring now to FIGS. 39 to 42 inclusive, there is shown an eighth embodiment of the present invention, indicated generally by the reference numeral 600, where like parts have the same reference numeral as before. The embodiment shown in FIGS. 39 to 42 inclusive differs from the other embodiments in that there is provided a vacuum cylinder 601 which may be attached onto the end of a body 603 to provide a vacuum source. The vacuum cylinder 601 is not a bellows and does not contract. It is envisaged that the vacuum cylinder may be provided by way of a capsule that has been evacuated and sealed. The body 603 has a needle 605 connected thereto however this is not essential, a separate needle may be connected to it. The body 603 further comprises an internal piston 607 housed in a cylinder 609 casing. The piston 607 is configured for movement in a direction parallel to the longitudinal axis of the body. The internal piston has an abutment surface 611 with a plurality of ribs arranged in a saw-tooth configuration along its length for engagement of an abutment arm (not shown) mounted inside the cylinder casing 609. The body 603 further comprises means to pierce a seal on a vacuum cylinder, provided by way of a hollow pipe 613, open at both ends, mounted on the outer end of the cylinder casing 609 and having a sharpened part for piercing the seal of the vacuum cylinder.

In use, once the needle tip has entered the patient, the vacuum cylinder 601 is introduced onto the end of the body 603 by piercing the vacuum cylinder 601 with the hollow pipe 613. The vacuum cylinder will in turn create a vacuum inside the cylinder casing 609, which will draw the piston inwardly. The piston will be held in position until such time that the needle tip enters into a blood vessel and the pressure at the needle tip drops. Blood will start to flow through the needle into the cylinder casing 609, the internal piston will be pulled rearwards towards the vacuum cylinder 601, and the abutment surface engaging the abutment arms will cause the audible and/or the haptic feedback.

It will be understood that there are a number of differences and advantages of this embodiment to the previous embodiments. First of all, the vacuum is applied with a source other than a bellows. Secondly, the vacuum source can be removed if a one way valve (duck bill valve for instance) is added (not shown). The magnitude of the vacuum can be selected for different patients physiology or for different tasks, depending on the level of vacuum deemed necessary by the physician, by having different vacuum cylinders with different levels of vacuum (measured in KPa) therein. This embodiment has the advantage that it is very small.

It is envisaged that the vacuum could be manually generated using a syringe, permitting regeneration of the vacuum following a failed insertion attempt, but a pre-loaded vacuum vial is deemed better. A push-on vial to introduce the vacuum pierces a rubber seal. A very small volume of air on the other side of the piston is present so it won't move much, and the volume of air is minimised by reducing any clearances between the main housing and the plunger and ensuring the audible/haptic feedback mechanism has the minimum possible air space in its enclosure. The piston flange is pressed so it directly engages the abutment surface. Advantageously, a wiper is used to limit friction on the plunger so that small pressure changes will register as “clicks”. Importantly, the internal diameter of the main housing is as small as possible to reduce the area of contact between the wiper and the internal walls, further minimising the friction. A small diameter of the main body will result in a higher vacuum being formed relative to the larger volume of the vacuum chamber. While only one row of ribs is shown, it will be appreciated that more than one row may be used with an increased number of abutment arms as previously described to increase the number of clicks for any movement of the piston.

While an abutment arm is described for generating the feedback, it will be appreciated that a plug could be inserted between the valve and vacuum chamber. In this embodiment, the plug maintains a seal between the air volume on the valve side and the vacuum chamber. When the vacuum is applied, preferably there is a small volume of air on the valve side of the plug which exerts pressure on the plug with a vacuum applied, the plug being releasably held in a seat by retaining means within a defined pressure differential either side of the plug. As before there is a minimal amount of air on the needle side of the valve to ensure that the plug is not released prior to fluid entering the needle. On opening of the valve and fluid entering the needle the additional pressure generated on the valve side of the plug releases the plug which is projected away at speed (due to the pressure differential) from its seat audibly contacting a surface to generate noise which indicates that blood has entered the needle. The plug could be a metal bearing and the contact surface another metallic surface generating a distinctive and highly audible sound. It will be appreciated that the movement of the plug on release could generate sound through clicking as described before.

While the introduction of the vacuum from the vial has been described above by way of piercing a rubber seal, it will be understood that the vacuum may alternatively be applied by opening a valve between the vial and the port to generate a vacuum at the port (as described in previous embodiments).

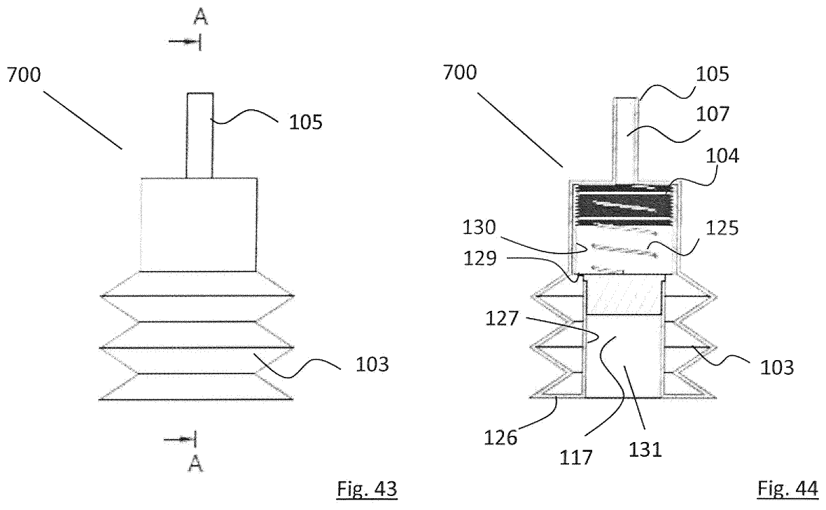

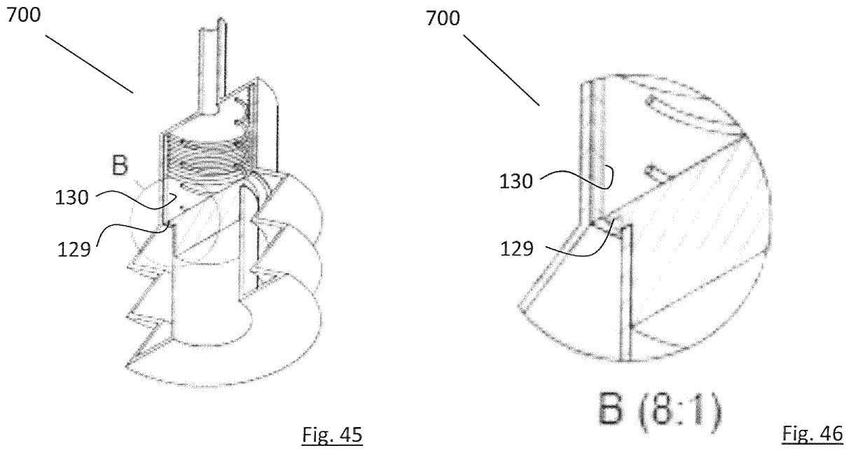

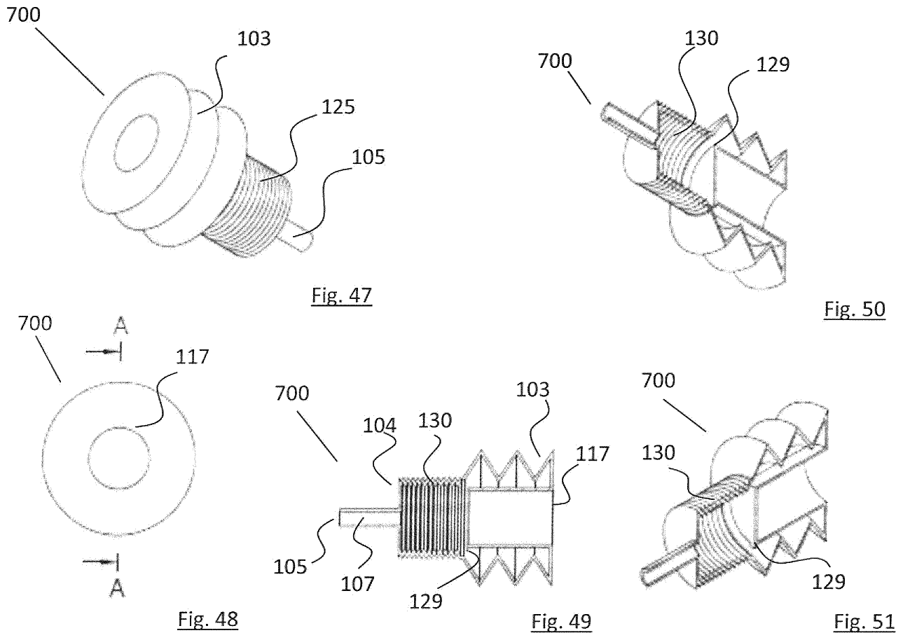

Referring to FIGS. 43 to 51 inclusive, there is shown a ninth embodiment of the present invention, indicated generally by the reference numeral 700, where like parts have been given the same reference numeral as before. This is a very simple implementation that will be inexpensive to manufacture and simple to use. The device 700 comprises a port 105 for attachment of a needle (not shown), a bellows 103 that may be compressed, and a spring 125 for aiding in the expansion of the bellows. A valve is not provided but could be if desired. An abutment arm 129 is mounted on the inside of the bellows recess 131 and an abutment surface 130 with ribs is formed on the bellows 104. The inner bellows 104 is compressed and evacuated of air by pressing in the plunger 117.

The embodiment shown in FIGS. 43 to 51 inclusive comprises a one-piece bellows and ribs arrangement. It has a very small air pocket and is mouldable in one part. In use, the user compresses the bellows to form the vacuum. The user maintains the bellows in its compressed state by squeezing the external annular surface 126 which holds the cylindrical shaft 127 in the bellows recess 131. On insertion of a needle (not shown) attached to the port of the device, the user releases their grip on the surface 126 which releases the shaft 127 thus applying a vacuum in the bellows as it expands once more and by extension, a vacuum to the port 105. The device could have a valve adjacent the port which operates in a similar manner to previous embodiments. The advantage of this solution is that the user feels the movement of the abutment arm 129 against the ribs 130 while the bellows expands.

The drawings show just one abutment arm 129 which runs over the ribs 130 in the main body of the device. The abutment arm could be an annular ring and the ribs may be formed circumferentially inside the body. Alternatively, the ribs of the abutment surface, the abutment arm and the spring may be added after moulding through an opening (not shown) in the end of the cylindrical shaft 127 located in bellows recess 131.

The cylindrical shaft 127 located in the bellows recess 131 serves to fill the air gap in the bellows when the bellows is compressed. The diameter of the cylindrical shaft 127 and bellows recess 131 is close to the collapsed internal diameter of the main body (i.e. the bellows chamber) and advantageously, is narrower in diameter. In this way, the ribs that receive the central component when the bellows is collapsed thus significantly reducing the air in the collapsed bellows. As a further alternative, the ribs could be on a sleeve which sits in the narrower section of the main body of the bellows, with the abutment arm on an insert as shown in the drawing. A spring provides additional vacuum/responsiveness as required.

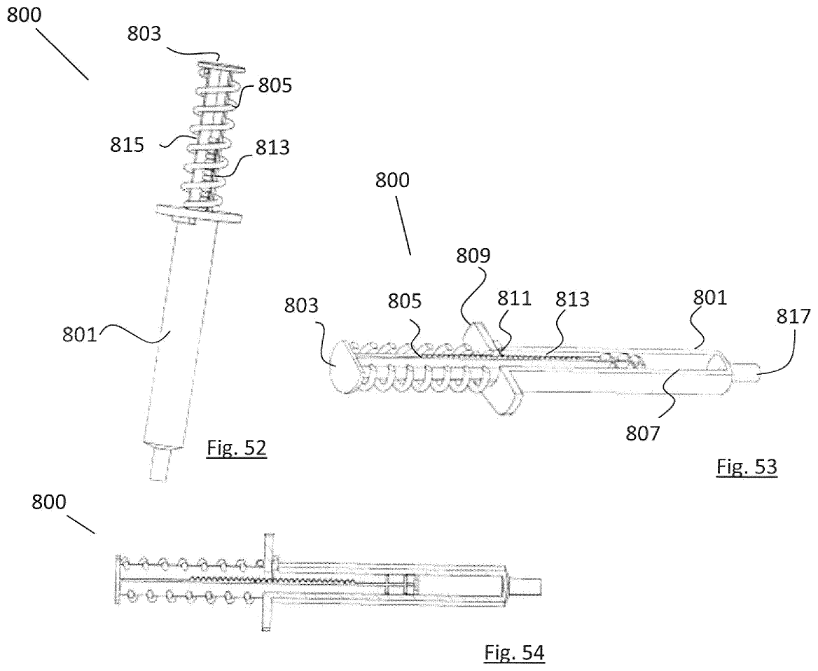

Referring to FIGS. 52 to 54 inclusive, there is shown a tenth embodiment of the present invention, indicated generally by the reference numeral 800, where like parts have been given the same reference numeral as before. The device comprises a body 801 akin to a standard syringe, with a plunger 803. The syringe is preferably a so-called “frictionless” syringe which are known in the art. A spring 805 biases the plunger outwardly thereby operable to draw fluid into the syringe chamber 807 and cause a vacuum internal the syringe chamber 807. A collar 809 has an abutment arm 811 thereon for engagement of an abutment surface 813 along the shaft 815 of the plunger 803.

A valve (not shown) may be added to the port or a release latch which controls the position of the plunger 803 in the body 801. In use the user depresses the plunger 803 and closes the valve (not shown) to maintain the spring 805 in a compressed state with the plunger 803 positioned such that there is a minimum amount of air in the chamber 807 between the plunger and port 817. The user inserts a needle (not shown) attached to the device into a patient and opens the valve to apply a pressure to the port. On the needle penetrating a vein, the spring forces the plunger to extend with the abutment surface 813 on the plunger moving past the abutment arm 811 clicking as it moves. While the use of this embodiment has been described as using a valve a latch may be deployed which automatically holds the plunger in a fully depressed position, the latch being released by the operator when the needle has been inserted into the patient.

There are several additional advantageous aspects of the different embodiments of the present invention and these will be outlined below:

The device is extremely light weight/does not interfere in the delicate manipulation of the needle by the operator. Of significance is the activation of the valve or latch which enables a vacuum at the tip of the needle but does not result in the unintended movement of the needle tip which can cause the operator to damage or miss a blood vessel. The third embodiment described above requires a rotation of the valve with the force being applied perpendicularly to the needle thus preventing the needle moving forward/backwards. By the fact that the needle is located in a patient when the valve is opened it can only move forwards or backwards. The general nature of the silicone valve being such that irrespective of the position of the lever it requires very little force to open. Similarly, for those embodiments with a latch arrangement, the operation of the latch will not negative impact on the positioning of the needle in the patient.

The design of the end of the device adjacent the needle with the valve lever does not interfere with the standard grip used by operators. A light-touch squeeze valve is described but other types of valve may be used as would be understood by the skilled addressee. Importantly, the valve/latch is adjacent where the operator holds the needle and is so easily accessed and actuated by the gentle movement of an operators finger.

Operators have developed the fine motor skills and technique/grip for directing a needle towards small blood vessels over many years, and it is an aim of this invention that it does not ask them to deviate from this. The device therefore needs to be lightweight and operable with the grip and movements they currently deploy. The means for deploying the vacuum at the needle tip need to be proximal the needle grip, easily found and require minimum travel and force to operate. Importantly in the embodiments shown the backwards sweep of the lever does not encourage the needle further into a blood vessel with the resulting risk of going fully through a blood vessel. The operator should not have to change or release their grip on the needle to release the valve with the valves being easily released with a normal grip on the needle or even on the device itself.

As mentioned above, it is important that the device does not require a change in how a user holds the needle. This is a learned method and cannot be compromised nor can they afford to stop mid-insertion to open a valve. The valve should be lightweight, the valve lever should be easily found and therefore needs to be proximal to the operators fingertip and its point of contact should be as far forward towards the needle as possible. Preferably the motion required to open the valve should not move the needle forwards or backwards but the valve should provide positive feedback in the open and closed positions. Valve release needs to be positive so that the user is confident that the device is primed and active. The valve can be of any type including a stopcock but its release particularly should not cause an operator to change grip on the needle or apply a force directly or indirectly on the needle which may result in an unintentional change in the needles position or trajectory relative to a blood vessel, resulting in an undesired outcome.

Preferably, the valve is provided by way of a light pinching of a silicone tube by a lever which only requires a light touch to remove the pinching in the tube and allow air initially and then blood to flow into the bellows.

The rebound energy of the bellows could also be increased by applying an external vacuum with the bellows/vacuum chamber located in a larger bellows/vacuum chamber. Other variations may be made without departing from the present invention. While the double bellows and spring have been shown in various embodiments, it will be appreciated that their use to increase the vacuum and rebound energy of the bellows to improve sensitivity may be used interchangeably in one or more of the other embodiments. Indeed, a spring may be used with the double bellows if required to generate a highly sensitive version of the device with a very high vacuum.

The pitch of the abutment surface against the pitch of the abutment arm can be important. Increasing the pitch of the ribs and increasing the number of abutment arms which do not click simultaneously on the ribs can be worthwhile. More than one row of ribs on a body, each row having its own abutment arm, and each row or abutment arm pair being arranged such that no click occurs simultaneously from each row/clicker pair while the bellows is expanding. Reducing the pitch of the ribs and the arms will provide for a higher resolution and will increase the chance of a rib being on the cusp of clicking when the valve or bellows are released thus providing for a quicker first click indicating the vein has been punctured. The smallest diameter of bellows practical to accommodate the means for generating haptic/acoustic feedback is preferred.

The abutment arm design is such that it needs to click quickly especially for the first click. Therefore, it is not too long. It also cannot be too stiff (due to a combination of material and thickness) or it will add friction and limit movement of the bellows potentially delaying the first click. In some embodiments, the abutment arm could be constructed from rubber, such as a rubber having a shore A hardness of the order of 40 A to 80 A. This has been shown to provide a good level of acoustic feedback and is flexible enough to limit friction on the expanding bellows. As stated previously the abutment arm could also be of thin plastic, metal or elastomer. In the embodiments described in FIGS. 1 to 14 inclusive, it is envisaged that the material of the arms and the ribs can be relative hard polymers, such as Acrylonitrile butadiene styrene (ABS) with a typical shore hardness of the order of greater than 60 Shore D.

Different sound effects can be generated by using different combinations of material in the arm and ribs. A rubber arm impacting against a plastic rib produces a low pitch and to be audible requires a relatively large contact area (the contact surface defined by the area of the rib that impacts the ribs) between the two and a relatively stiff arm to create sufficient volume to be heard through the bellows and felt by the operator, this relatively large contact area and stiff arm adds resistance that has to be overcome by the spring and or bellows which can make the plunger difficult to depress for some users and make the device less responsive to pressure change. The same is true with a stiff plastic or metal arm, while capable of generating an audible sound with a smaller contact area this combination still generates relatively high friction which typically needs to be overcome through the use of a spring.

Advantageously the arm can be a combination of hard and flexible material with the more flexible material at the base or it may be entirely of a hard material and fixed in a flexible base to allow for the relative ease of bending to reduce resistance to movement as previously described. The arrangement in the first and second embodiments is different in that it deploys a longer lever to generate the acoustic and haptic feedback. It has been found that having a lever and plunger of ABS polymer with a shore hardness of minimum 60D provides the required haptic and acoustic feedback even when immersed in blood.

It has been found through experiment that providing the pitch of the ribs at approximately 1 mm allows the arm to build up enough speed once it leaves the preceding rib to make an audible sound when striking the next rib. Reducing this pitch below 1 mm can reduce the kinetic energy in the arm and hence the impact force and sound generated from impact.

It will also be appreciated that the effective amount of strike surface area between the rib and arm can be changed by adding a radius or chamfer to the impacted corner of a square shaped rib increasing the audible volume of the impact making the clicking more audible. It will be appreciated that the pitch and height of the ribs could be varied to provide different sounds as the arm(s) move relative to the ribs indicating various stages of the bellows expansion and contraction. For example there could be a larger gap between the first rib to be impacted on release of a latch or valve and the next rib, with the first click indicating the device is primed and the second click indicating blood entering the needle. This larger gap allowing for the expansion of the bellows on release of the compressing force but before blood enters the needle. The larger gap also indicating the bellows was fully collapsed. The pitch and amplitude could also be varied to indicate that the bellows has been fully collapsed. This will ensure the maximum vacuum is achieved in the bellows and that it has sufficient travel to provide the device sensitivity.

It will be appreciated that the arm(s) can be hinged to provide different resistance in different directions and hence different sounds. Indeed a torsion spring could be usefully deployed to achieve this, providing a very high level of kinetic energy on release from a rib with an object of hard material (or the spring could be locally hardened) on the strike end to make an audible striking sound on the ribs, the spring providing different resistances depending on the direction of bending. The torsion spring could also be used to control a stiffer rib. Needless to say other types of springs could be employed such as a helical spring at the base of a stiff rib, or a leg of a helical spring acting as an arm. In all cases the helical spring provides the flexibility required to produce the required kinetic energy in the rib while minimizing friction.

While the bellows is generally shown as being in line with the needle it will be appreciated that the bellows may be at an angle of up to 90 degrees to the needle, effectively shortening the overall length of the device which will allow an operator to easily hold the device between thumb and finger. This effective shortening of the device importantly reduces the chance of the device touching an operator's palm, especially when the bellows is extending thus not requiring the operator to change a well established fine motor grip which is critical for the careful guidance of the needle in operation, this being especially relevant for operators with smaller hands. This provides a significant advantage when compared to the traditional use of a syringe which does require an operator to change their grip, the typical syringe employed being longer than the device.

It has been found that the fluid entering the bellows can flow over the ribs and arm arrangement in some embodiments and even cause these to be submerged. It will be appreciated that a channel can be provided in the fluid passageway opening or plunger to encourage the initial fluid away from the arm and ribs as it comes into contact with the plunger and fills the bellows to minimise this damping effect so that the clicking sound is as loud as possible and especially audible for the initial few clicks before the bellows fills with fluid. Alternatively a low friction seal could be added to the free end of the plunger, which moves with the plunger in the fluid channel to prevent blood immersing the first ribs as they are impacted by the arm(s) further limiting the acoustic dampening of blood. To delay the contact of fluid with the arm and ribs the ribs and arm can be positioned such that they are at the highest point and away from the initial fluid ingress into the bellows.

While an arm has been described as rubbing against ribs to provide for acoustic and audible feedback it will be appreciated that any rubbing caused by the moving bellows in response to a reduction in the vacuum or ingress of blood can provide acoustic/haptic feedback. Advantageously the ribs provide for a low friction solution maximising the available force in the bellows for generating a vacuum and also provide for very effective audible feedback. While the bellows has been shown to be cylindrical in shape it will be appreciated that its shape may vary, indeed it may have a square or rectangular cross section. Any air pocket between the needle and bellows should be minimised if possible such that any bore connecting the needle and bellows should be preferably no larger in diameter than the diameter of the largest needle that is expected to be used on the device or only as small as is practical for manufacturing.

While the bellows has been described as generating a vacuum by virtue of its own wall stiffness, it will be appreciated that the vacuum may be generated by applying an extending force by use of a spring or other means to expand its volume. The double bellows may have a helical fold to enable the release of the inner core from the mould. The expansion of the bellows with such a helical pattern of folds could result in a rotation along its axis as well as an extension. The bellows is preferably blow moulded but may be compression or rotationally moulded. Where the opening of the bellows is large enough in relation to diameter, the bellows may be injection moulded if the material has a high elastic limit.

Preferably, it is desirable to have a small bellows volume so for a given volume change it is possible to achieve the largest possible dimensional change in the bellows and to reduce the weight of the device. The device should be able to draw enough blood into a transparent/opaque channel just after the needle so the operator can see this blood to confirm visually that the needle is properly in place for additional confidence. The required vacuum to draw blood will depend, at least in part, on the needle gauge, and this may be catered for accordingly. In some embodiments the bellows moves against the user's hands (palm) or a triggering mechanism may be provided to touch the users hands which could also be used as an indication that the needle has punctured a blood vessel successfully.

It will be appreciated that the plunger's movement could be converted to an electrical charge which could power a sound or light source to alert the operator of the bellows extensions. For example the abutment arm could be a piezo strip which, while being flexed generates a voltage which could be employed as an example to make an LED light up, power a buzzer or other sensor or indicator which could further alert the operator of movement of the bellows.

Where not specifically described, springs could be applied either internally or externally to any of the embodiments. Where an abutment arm has been shown separate to a bellows in many cases, it will be appreciated that it could be part of the bellows. Indeed, if part of the bellows it will preferably be placed at the free end of the bellows but it may be located anywhere on a moving portion of the bellows if desired.

It has been established that the bellows preferably needs to be able to generate a sufficient vacuum in the ranges of 150 mmHg (20 kPa) and 250 mmHg (33.3 kPa) at the tip of a range of needles ranging from Gauge 16-22, having a length of between 3 cm (0.03 m) to 8 cm (0.08 m). When the needle is connected to the bellows, and when the bellows is fully compressed to pull blood up through a needle, it needs to have minimal amount of air in its chamber when fully compressed to avoid any increase in pressure, causing a reduction in volume (according to Boyles Law) and it needs to be able to provide a sufficient dimensional change to enable the generation of acoustic and/or haptic and/or visual feedback when blood enters the device.

It has been found that using a spring with a rate of 1635 Nm provides a vacuum of 180 mmHg in the third embodiment and provides for a rapid response using a variety of needles.

It is critical that the vacuum is present at the needle tip prior to entering the blood vessel so that on entry to the vessel the vacuum is released in the bellows chamber and this registers as a change of dimensions in the bellows which results in a click or other positive acoustic and/or haptic feedback. Having the abutment arm on the end of the bellows strained against a rib for example will ensure the first important click occurs as rapidly as possible. It is not necessary that blood travels the full length of the needle but that while travelling in the needle a pressure change is caused in the bellows which is registerable. Indeed, it is preferable that the first click occurs more quickly than blood would passively exit a needle, were the needle not connected to a device, as this will provide faster feedback to the operator than the traditional visual ‘flashback’ method, notwithstanding that an operator must look away from an ultrasound screen to avail of this traditional cue.

To maximise the sensitivity of the device the design needs to translate the smallest pressure increase in the system into movement of the bellows to provide the important first clicks. To enable this, the bellows needs to have a chamber volume (when the bellows is compressed and with a vacuum generated) such that a small volume of blood in the needle will translate as a measurable dimension change such that a haptic/acoustic signal can be generated by the abutment arm. The bellows needs to be able to generate the required vacuum while depressed to pull the blood into the needle as rapidly as possible to cause this dimension change in the bellows as quickly as possible.

There is therefore a need for a bellows design that can provide the required vacuum at the needle tip of commonly used needles so that the bellows can pull blood through the needle, and that this blood in the needle will provide for an adequate dimensional change in the bellows, with the resulting pressure increase such that this dimensional change will result in an acoustic/haptic feedback more quickly than flashback would ordinarily provide. While it is preferable that this occurs more quickly than flashback, it will be appreciated that even in the event that the auditory click is heard at the same general time as flashback is observed, the auditory feedback removes the need for the operator to take their attention off the needle tip and will ultimately provide for quicker feedback and not distract the operator from viewing the screen, hence removing the risk of the operator moving the needle too far or veering off target.

Through approximation, it is understood that a 16 gauge needle @ 5 cm long will provide a displacement of the order of 1.3 mm with the introduction of 0.1 ml of fluid in a cylindrical bellows of internal compressed diameter 5 mm and a compressed volume of 1.26 ml, with no airpocket when compressed, which will be enough movement to provide for acoustic feedback in the embodiments described (where the ribs are spaced out at pitches of at least less than the displacement). Bearing in mind that the bellows will increase the rate of blood flow through the needle this will provide for an improvement in the time in which an operator will receive notice of the correct placement of a needle over the traditional passive flashback method. A bellows with less volume will provide proportionally more movement which will provide more resolution and will be even more superior in terms of the speed of notice to the operator. An operator may want to use the device to sample blood so a larger uncompressed bellows volume of circa 2.5 ml may be preferable.

The device also allows for the collection of blood for analysis with the bellows in at least some of the embodiments being removable from the main body of the device as required, or the blood may be expressed from the device by compressing the bellows—this extended use may necessitate the addition of an anti-coagulant. While the abutment arm has been described as moving over fine pitched ribs, it will be appreciated that it may move over a rough surface or bumps or through a tortuous path (where the abutment arms are provided by way of protrusions or bumps) which would provide suitable haptic feedback. While the bellows has generally been described as directly moving a clicker/abutment arm against ribs or a rough surface, it will be appreciated that it may directly move the ribs or rough surface along a rib(s). Indeed, it may cause the ribs/rough surface to move indirectly over a clicker(s)/abutment arm(s) which may be connected to or part of a bellows.

As mentioned above, it is important that when fluid enters the device that the corresponding reducing vacuum level in the system is translated into the maximum dimensional change in the bellows. Any deformation in the bellows, when being physically depressed to create a vacuum in the bellows that does not directly translate into an acoustic/haptic/visual feedback on recovery, should therefore be preferably avoided or minimised. Bellows manufactured from low density polyethylene (LDPE), polypropylene, Hytrel®, thermoplastic elastomers or other thermoplastics have wall thicknesses that are generally in the range of 0.1-0.3 mm, easily allowing for deformation, or of softer silicone or TPE material that deform easily. Were there an air pocket present on the full depression of the bellows, this would further delay and reduce the bellows movement such the user would question the value of using the device. For example, if the depression caused by the concave end was 1 mm based on the prior example the recovery of this depression would seriously impact the movement of the bellows as the movement was only 1.3 mm with 0.1 ml of fluid entering the system (which it is understood is approximately equivalent to twice the volume of a 16 Gauge needle @ 5 cm long). Providing means to allow an operator to prevent creating such deformations is desirable and could include a second non-deformable surface that engages with the bellows to avoid said deformation. Preferably the bellows could be affixed to said surface to ensure that the only deformation of the bellows occurs at the fluted walls and nowhere else.

The extension in the bellows is also not even as the bellows does not extend along its axis due to the flexible nature of the bellows with the bellows bending as it extends (ends are not parallel). Guiding means may be provided to ensure the movement of the bellows is maximised for any vacuum reduction. This guiding means is preferably provided by a spring directing the bellows in a particular direction as other guiding means may introduce friction into the system. Springs, as described in the embodiments above, are provided to also encourage an even expansion of the bellows along the length where the abutment arm/rib arrangement requires.

It may however be preferable to encourage the bellows to bend and extend more on one side. By limiting the ability of one side of the bellows to move, this encourages further movement of the free side, producing a larger displacement that can be translated into feedback for a given vacuum reduction. In this way, this allows for an earlier first click for a given volume of fluid entering the device. Advantageously, the restricted side may be released by an operator to allow for the bellows to fully extend once the needle is confirmed to be in place, or the restricted side of the bellows may automatically release due to its changing angle as it extends. Referring to the third embodiment described above, the ribs may have different heights, thus releasing more easily on one side, allowing relatively more movement on the other side which provides increased resolution. The ribs on the freer side could be positioned to maintain contact with the abutment arm as the bellows bends.

As described above, air pockets in a fully depressed bellows prior to the valve being closed create delays in the expansion of the bellows and therefore delay the first “clicks” of audible and/or haptic feedback. This is due to the fact that the pressure increase compresses the air (P1V1=P2V2) and contracts the bellows (which we want to expand as quickly as possible). To reduce the air pocket, one or more of the following techniques could be used:

First of all, it is preferable that the ends (walls without folds) of a cylindrical bellows are mostly parallel to each other so that they touch, or are as close to each other as practicable, when collapsed. Secondly, a thin bellows material is used that will allow for the maximum compression of the bellow folds. In this way, the stacked heights of the folds in the walls is minimized, thereby reducing the dead space between the stacked walls. Thirdly, eliminating the airspace between the stacked bellow walls by filling the volume with a protrusion in the bellow itself, or by a separate part which could include means for connecting to a valve or directly to a needle, is seen as useful. Fourth, a double bellows as described whereby the protrusion described above is effectively a second inner bellows incorporated into the first outer bellows. Fifth, the fluid channel between the main bellows chamber and the valve can be partially filled to reduce this volume of air, leaving enough space to allow fluid and air to pass but limiting the air pocket. Sixth, the partial filling of the fluid channel could be applied between the valve and the opening at the needle hub where blood enters the device. All or some of these techniques could be used to good effect to reduce the air pocket size inside the bellows.

It is envisaged that the volume of the fluid passageway between the opening of the needle hub and the point of closure of the valve is less than 0.05 ml and preferably smaller (actual design has it as 0.02 ml). On valve opening, there will be a predefined number of clicks allowing for the air in the needle and the needle side of the valve to affect a vacuum reduction in the bellows before the needle meets fluid, thus confirming activation of the device to the operator. It may be preferable to arrange the spacing of the abutment arms and abutment surfaces such that there are no initial clicks on opening of the valve prior to entering the blood vessel to eliminate false positives.

It is envisaged that various embodiments may be used for the generation of haptic feedback. First of all, the arrangement for haptic feedback may be applied at the outside diameter of the cylindrical bellows. The bellows has a disc either formed or attached, the disc preferably being of a thin material in the range of 0.1 mm (0.0001 m) to 1 mm (0.001 m) thick that can flick over ribs. The ribs are positioned proximate the outside surface of the bellows such that the disc flicks over the rib surfaces when the bellows expands, thereby providing feedback. A plurality of rows of ribs could be provided. Preferably, if multiple rows of ribs are provided, they will be equally spaced around the bellows to reduce the tendency of the bellows to bend, and potentially get wedged while expanding, as this would cause the device to stop pulling blood into the bellows and stop providing haptic feedback.

Where a spring is deployed, sound and or haptic feedback may be generated by movement of any portion of the spring against an abutment arm or other protrusion.

In the embodiments described, reference is made to an abutment arm, or an abutment disc that engages against the abutment surface. It will be appreciated that the abutment arm may be a separate and distinct arm or a plurality of separate and distinct arms, the arm may be an elongate arm, or the abutment arm may take the form of a disc, a protrusion, a plate, a collar or an annular ring mounted on one of the body, the vacuum chamber or the bellows (if provided). The disc, plate, collar or annular ring may have a uniform outer circumference, or may have one or more circumferentially spaced protruding tabs for engagement of an abutment surface. The abutment arm may be a rough surface.

Secondly, the arrangement for the haptic feedback could be applied along the longitudinal axis of the medical device. In this way, the bellows pushes a ribbed shaft over a flicker (or vice a versa), the ribbed shaft preferably aligned with the bellows longitudinal axis. It will be understood that where the clicker/ribs are in contact with fluid as it enters the bellows, that their position is such that fluid may pass by without impacting their function. However, preferably, they are positioned so that fluid does not pass over them during entry into the bellows (when the device is held in the intended orientation) and they are not located where fluid will gather initially in the bellows therefore not hampering their ability to generate the important first clicks. The location of the abutment arm/ribs is such that the vibration that is caused by their engagement is felt through the various housings by the operator. It is envisaged that when the abutment arm/clicker and abutment surface/rib combination is internal in the bellows, the sound waves will dwell for longer and will therefore provide a longer signal of feedback to the operator.

The priming of the device after entry into the body and before entry into the blood vessel may be achieved in a number of ways. First of all, by opening of a valve to apply the vacuum in the bellows to the needle tip. Secondly, by physically releasing the contracting force on the bellows or spring to allow the bellows or spring to expand under their own force, applying a vacuum to the needle tip. And thirdly, by applying an extending force to a contracted bellows separately or in combination with the first and second methods described above.

A one-way valve may be fitted to the bellows to provide a release for air should a user mistakenly depress the bellows while the needle is in a patient. It will be understood that in all of the embodiments described, the device will prevent air being drawn into the patient's bloodstream (air embolus) which is a potentially lethal complication that could arise if a needle or catheter-over-needle device is used for initial central venous puncture.

While the present invention is described in terms of an operator using ultrasound guidance, it will be understood that the present invention may also be used to find a vein generally without ultrasound, for example in a general clinical setting for finding blood vessels. Furthermore, the device may be applied to a number of alternative procedures such as, but not limited to; biopsies, fine needle aspiration, bone marrow aspiration and placement of chest or peritoneal drains by Seldinger technique among others.

In this specification, the term resiliently deformable has been used. It will be understood that by resiliently deformable, what is meant is a property that will cause a material that has been deformed out of its natural shape, to return to its original shape once the force is removed.

In this specification the terms “comprise, comprises, comprised and comprising” and the terms “include, includes, included and including” are deemed totally interchangeable and should be afforded the widest possible interpretation.

The invention is not limited to the embodiments hereinbefore described but may be varied in both construction and detail within the scope of the appended claims.

1) A medical device for use in aiding the successful placement of a needle tip in a blood vessel, the medical device comprising a body defining a vacuum chamber therein, the body having a port for engagement of a hub of a needle, the port defining a fluid passageway from the exterior of the body to the vacuum chamber in the interior of the body, and means to generate at least one of an audio feedback and a haptic feedback to a user acted upon by a vacuum in the vacuum chamber; and in which the means to generate the feedback comprises an abutment arm and a complementary dedicated abutment surface operably engaged by the abutment arm; the abutment surface having a plurality of closely-arranged spaced abutment members disposed along its length and opposing the abutment arm; the vacuum in the vacuum chamber being operable to move one of the abutment arm and the abutment surface relative to the other of the abutment arm and the abutment surface, thereby causing the abutment arm to releasably engage at least one of the abutment members of the abutment surface to produce the feedback.

2) The medical device as claimed in claim 1 in which the abutment arm and the abutment surface are located internal the body.

3) The medical device as claimed in claim 1 in which in which there is provided a valve in the fluid passageway intermediate the exterior of the body and the vacuum chamber operable to selectively apply the vacuum to the port.

4) The medical device as claimed in claim 1 in which there is provided a releasable latch operable to control the application of the vacuum to the port.

5) The medical device as claimed in claim 1 in which the vacuum chamber comprises an elongate collapsible bellows.

6) The medical device as claimed in claim 5 in which the collapsible bellows is constructed from a resiliently deformable material.

7) The medical device as claimed in claim 5 in which there is provided a spring operable to extend the collapsible bellows.

8) The medical device as claimed in claim 5 in which the bellows has a pair of substantially planar opposing end plates.

9) The medical device as claimed in claim 5 in which the bellows is provided with an internal plug operable to substantially fill the void internal the bellows and minimize the amount of air inside the bellows when the bellows is in a collapsed configuration.

10) The medical device as claimed in claim 5 in which there is provided a guide means for the bellows to control the direction of travel of the outermost end of the bellows and by extension the shape of the bellows as it transitions to or from an expanded configuration to or from a contracted configuration.

11) The medical device as claimed in claim 5 in which the bellows is dual skinned with an inner bellows and an outer bellows.

12) The medical device as claimed in claim 5 in which the bellows is substantially cylindrically shaped.

13) The medical device as claimed in claim 1 in which the vacuum chamber comprises a releasably detachable vacuum chamber.

14) The medical device as claimed in claim 1 in which there are provided a plurality of abutment arms.

15) The medical device as claimed in claim 14 in which there are provided a plurality of complementary abutment surfaces.

16) The medical device as claimed in claim 15 in which there is provided a dedicated abutment surface for each abutment arm.

17) The medical device as claimed in claim 3 in which the valve is actuated by applying a force perpendicular to the longitudinal axis of a needle connected to the body.

18) The medical device as claimed in claim 4 in which the releasable latch is actuated by applying a force perpendicular to the longitudinal axis of a needle connected to the body.

19) The medical device as claimed in claim 3 in which the fluid passageway is defined by a section of the body constructed of silicon rubber for engagement by the valve.

20) The medical device as claimed in claim 1 in which the vacuum is between 150 mmHg (20 kPa) and 250 mmHg (33.3 kPa).

21) The medical device as claimed in claim 1 in which the valve has a shore hardness of between 10 and 55 Shore A.

22) The medical device as claimed in claim 1 in which the volume of the fluid passageway between the opening of the needle hub and the point of closure of the valve is less than or equal to 0.05 ml.

23) The medical device as claimed in claim 1 in which the port is provided with a side port branching off therefrom, the side port having a closure thereon.
